(19) United States
(12) Patent Application Publication
(10) Pub. No.: US 2009/0181003 A1
(43) Pub. Date: Jul. 16, 2009

(54) Regulation of S6 Kinsase Protein Activity and Related Methods

(76) Inventors: Ivan Gout, London (GB); Timothy Robert Fenton, San Diego, CA (US); Heba Ismail, London (GB)

Correspondence Address: MORGAN LEWIS & BOCKIUS LLP, 1111 PENNSYLVANIA AVENUE NW, WASHINGTON, DC, 20004, US

(21) Appl. No.: 11/990,226

(22) PCT Filed: Aug. 8, 2006
(86) PCT No.: PCT/US2006/030675

§ 371 Date: Feb. 13, 2009

Related U.S. Application Data
(60) Provisional application No. 60/706,010, filed on Aug. 8, 2005.
(60) Provisional application No. 60/785,981, filed on Mar. 27, 2006.

(51) Int. Cl. A61K 38/45; C12N 9/10; C12N 15/11; G01N 33/53; C12Q 1/48; C12N 9/99; C07K 16/18; C12Q 1/68

(52) U.S. Cl. 424/94.5; 435/193; 536/23.2; 435/7.8; 435/15; 435/184; 530/387.9; 435/6

(57) ABSTRACT

The invention encompasses agents and their methods of use for modulating the activity of kinases by effecting their acetylation or their binding to nucleic acids. The invention thus encompasses the modulation of S6 kinase by effecting its acetylation by p300. The invention further encompasses the modulation of S6 kinase 2 by affecting its binding to DNA.

### RELATED APPLICATIONS

This application claims priority to U.S. Provisional Application 60/785,981 (filed on Mar. 27, 2006) and U.S. Provisional Application 60/706,010 (filed on Aug. 8, 2005) both of which are incorporated by reference in their entirety.

### FIELD OF THE INVENTION

The present invention relates to methods of modulating protein kinase activity by an acetylating agent, to methods of treating and/or preventing protein kinase-related disorders and to molecules that catalyze the acetylation of protein kinases. The present invention further relates to methods of modulating protein kinase activity by binding to nucleic acids, to methods of treating and/or preventing protein kinase-related disorders and to molecules that can block DNA/protein kinase interactions.

### BACKGROUND OF THE INVENTION

The 40S ribosomal protein S6 kinases (S6Ks) belong to AGC family of serine/threonine protein kinases, which includes key players in the regulation of cellular functions such as PKB/Akt, PKA, PDK1 and PKC. There are two isoforms of S6K, termed S6K1 (also known as p70α S6 kinase or S6Kα) and S6K2 (also known as p70β S6 kinase or S6Kβ), which have cytoplasmic and nuclear splicing variants. The 23- and 13-amino-acid extensions at the N-termini of S6K1 and S6K2 contain nuclear localization signals (NLSs) that target these isoforms constitutively to the nucleus. The cytoplasmic form of S6K1 (S6K1-II) is predominantly cytosolic in serum starved cells, but translocates to the nucleus upon mitogenic stimulation. The presence of an additional nuclear localisation signal at the C-terminus of the cytoplasmic form of S6K2 (S6K2-II) determines its predominant nuclear localisation. Nucleaocytoplasmic shuttling has been reported for both cytoplasmic forms of S6K in cellular response to mitogenic stimuli, but the mechanisms of its regulation have not been elucidated.

S6 kinases are activated through mitogen- and nutrient-mediated pathways. Growth factor-activated receptor tyrosine kinases recruit PI3K, which, via its effectors PKB/Akt and PDK1, mediates S6K activation (Chung et al. (1994) Nature 1370, 71-75). Another major player in the activation of S6K is the mammalian target of rapamycin, mTor (FRAP) which senses the level of amino acids and possibly other nutrients within a cell (Hara et al. (1998) J. Biol. Chem. 273, 14484-14494). Mitogen- and nutrient-induced activation of S6K occurs through a complex series of serine/threonine phosphorylation events and requires precise interplay between specific domains and multiple phosphorylation sites (Dennis et al. (1998) J. Biol. Chem. 273, 14845-14852; Pullen and Thomas (1997) FEBS Lett. 410, 78-82; Weng et al. (1998) J. Biol. Chem. 273, 16621-16629.). Based on numerous studies a model for S6K activation has been proposed which implies that active conformation of the kinase is achieved by coordinated phosphorylations at regulatory residues clustered in the carboxyl-terminal autoinhibitory segment, the conserved kinase extension domain and the activation loop of the catalytic domain (Alessi et al. (1997) Curr. Biol. 7, 776-789; Dennis et al. (1998) J. Biol. Chem. 273, 14845-14852; Pullen and Thomas (1997) FEBS Lett. 410, 78-82). Regarding this model, the process of mitogen-induced S6K activation can be divided into several steps, regulated by divers signaling inputs. The initial step involves the action of an array of proline-directed kinases which phosphorylate the S6K carboxyl-terminal segment (Ser434, Ser441, Thr444, Ser447 and Ser452 in S6K1), and perhaps Ser394 in the linker domain (Price et al. (1991) Mol. Cell. Biol. 11, 5541-5550). This disrupts the inhibitory interaction between the carboxyl and amino termini and subsequently releases the phosphorylated carboxyl-terminal autoinhibitory segment from the catalytic domain, providing access to Thr412 and Thr252. The activation loop phosphorylation sites, Thr252 and Thr412, in the kinase extension domain of S6K1, are common regulatory elements found in most members of the AGC family of serine/threonine kinases (Hanks and Hunter (1995) FASEB J. 9, 576-96; Marshall, 1994). The kinase responsible for phosphorylation of Thr252 in vivo has been identified as PDK1 (Alessi et al. (1997); Pullen et al. (1998) Science 30 279, 707-710; Williams et al., 2000), which is know to phosphorylate several other substrates, such as PKB, PKCs and PKA. The identity of the kinase which can phosphorylate S6K at T412 is still unclear. It has been recently shown that Thr 412 in S6K1 can be directly phosphorylated by the mTOR kinase in vitro and that this phosphorylation induces substantial increase in S6 kinase activity (Burnett et al. (1998) Proc. Natl. Acad. Sci. USA 95, 8351-8356; Isotani et al. (1999) J. Biol. Chem. 274, 34493-34498). Subsequent phosphorylation of S6K1 by PDK1 gives a strong synergistic increase in S6 kinase activity (Burnett et al. (1998)). In addition, the NIMA-related kinases NEK6 and NEK7 have recently been identified and shown to phosphorylate Thr412, as well as some other sites, and activate S6K1 in vitro and in vivo, in a manner synergistic with PDK1 (Belham et al. (2001) Curr. Biol. 11, 1155-67).

Most of the phosphorylation sites identified in S6K1 are conserved in S6K2 and include: a set of (Ser/Thr)-Pro motifs clustering in the autoinhibitory sequence and region located immediately carboxyl-terminal to this sequence (Ser423, Ser430, Ser436 and Ser441 in S6K2-I, which correspond to Ser434, Ser441, Ser447 and Ser452 in S6K1-I, respectively); Thr379, Ser383, Thr401 and Ser416 located in the kinase extension domain, which correspond to Thr390, Ser394, Thr412 and S427 in S6KαI, respectively; and Thr241 located in the catalytic loop, which corresponds to Thr252 in S6K1 (Gout et al. (1998) J. Biol. Chem. 273, 30061-30064). However, one of the known S6K1 regulatory residues, represented by Thr444, is not conserved and substituted to valine in S6K2. Based on a comparison of the phosphorylation sites between these two kinases, it has been predicted that S6K2 might be activated through mechanisms which are similar, but not identical, to those proposed for S6K1 (Gout et al. (1998) J. Biol. Chem. 273, 30061-30064; Shima et al. (1998) EMBO J. 17, 6649-6659).

S6K is inactivated by complexing with protein phosphatases, such as PP2A and PP1 (Andres et al. (1987) J. Biol. Chem. 262, 14389-14393; Peterson et al. (1999) Proc. Natl. Acad. Sci. USA 96, 4438-4442). This interaction leads to dephosphorylation and subsequent inactivation of S6K. Two tumor suppressors, PTEN and TSC1/2 complex, are known to inhibit indirectly the activity of S6K. PTEN is a lipid phosphatase, which reduces the level of PIP3, a key mediator of PI3-K signaling pathway (Sansal et al. (2004) J. Clin. Oncol. 22, 2954-2963). The activation of tumor suppressor complex TSC1/2, a negative regulator of mTOR, leads to full inactivation of S6K (Harrington et al., (2004) J. Cell. Biol. 166, 213-23).

Both kinases share a high level of homology between their kinase domains, but differ significantly in the N- and C-terminal regulatory regions. It is believed that conformational changes induced by multiple S/T phosphorylations open the structure of S6K, making both N- and C-terminal regulatory domains available for protein-protein interactions. The list of S6K-binding partners includes small GTPases Rac1 and cdc42, protein kinases PDK1 and PKC zeta, protein phosphatase PP2A and PP1 and cytoskeletal protein neurabin.

Ribosomal protein S6 is the most widely studied physiological substrate of S6K. The phosphorylation of S6 protein was shown to closely correlate with the initiation of protein synthesis induced by various extracellular stimuli. Phosphorylation of IRS-1 by S6K was shown to be essential to multiple pathways that make cells unresponsive to insulin, including those initiating from continual exposure to insulin or free fatty acids (Giraud et al. (2004) J. Biol. Chem. 279, 3447-3454). The transcriptional activator CREM, elongation factor 2 kinase, the regulator of apoptosis Bad 1 and cytoskeletal protein neurabin have also been shown to be phosphorylated by S6K in vitro and in vivo (de Groot et al. (1994) Cell. Signal. 10, 619-628; Harada et al. (2001) Proc. Natl. Acad. Sci. USA 98, 9666-9670; Wang et al. (2001) Biochem J. 358, 497-503; Burnett et al. (1998) Proc. Natl. Acad. Sci. USA 95, 1432-1437). However, the physiological relevance of these phosphorylations requires further investigation, since other protein kinases can phosphorylate these molecules at identical sites.

Studies from numerous laboratories indicated that S6Ks are key players in ribosomal biogenesis and protein biosynthesis. The expression of translational components which make up the protein biosynthetic apparatus is controlled via S6K pathway (Thomas (2002) Biol. Res. 35, 305-313). In addition, a functional link between mTor/S6K signaling and Coenzyme A biosynthesis has been recently established (Nemazanyy et al. (2005) FEBS J. 272, E1-046P). Cell cycle progression requires S6K function at G1/S and G2/M phases (Banatti et al., 1998). S6K was found to be involved in regulating the expression of genes essential for cell cycle progression. In addition to playing an essential role in regulating growth and cell cycle progression, S6K1 appears to be a multifunctional protein involved in other cellular processes such as cell survival and RNA processing. These links are mediated through phosphorylation of anti-apoptotic protein Bad1 and complexing with SKAR protein, which has been proposed to couple transcription with pre-mRNA splicing and mRNA export (Harada et al. (2001) Proc. Natl. Acad. Sci. USA 98, 9666-9670; Richardson et al. (2004) Curr. Biol. 14, 1540-1549). So far, there are only sporadic data which implicate S6K in the regulation of transcription. It was reported that transcriptional activator CREM is the substrate for S6K in vitro and in vivo (de Groot et al. (1994) Cell. Signal. 10, 619-628). In addition, the function of transcription factor E2F was found to be regulated by mTor/S6K pathway in lymphocytes (Brennan et al. Mol. Cell. Biol. 19, 4729-4738).

Much of what is known about the physiological roles of S6Ks has come from genetic studies in Drosophila and mice. Drosophila possess only one S6K gene, dS6K, the disruption of which results in the death of most flies at the larval stage or during early pupation (Montagne et al. (1999) Science 285, 2126-2129). Those S6K1^{-/-} flies that do survive, live for only a few weeks and the females are sterile. These flies are also much smaller than wild type and this reduction in body size is due almost entirely to a reduction in individual cell size rather than cell number (Montagne et al. (1999) Science 285, 2126-2129). Therefore, it appears that a major physiological role of dS6K is as a positive regulator of cell growth rather than cell proliferation. Indeed, studies in which dPI(3)K or dPKB were knocked out again produced small flies, but a deficit in cell number was also seen, suggesting that the pathway controlling cell growth and proliferation bifurcates downstream of PI(3)K/PKB but upstream of dS6K (reviewed in Weinkove and Leevers (2000) Curr. Opin. Genet. Dev. 10, 75-80).

Disruption of the S6K1 gene in mice also produced a small size phenotype. This was again largely due to a defect in cell growth rather than in proliferation, although a slight reduction in the rate of proliferation was observed during development (Shima et al. (1998) EMBO J. 17, 6649-6659; Kawasome et al. (1998) Proc. Natl. Acad. Sci. USA 95, 5033-5038). As mentioned earlier, in S6K1 knockout mice, S6K2 expression is upregulated and appears able to restore phosphorylation of rpS6 to levels close to those of wild type mice in all tissues examined (Shima et al. (1998) EMBO J. 17, 6649-6659). This indicated that phosphorylation of rpS6 is not the mechanism by which S6K1 exerts its positive effect on cell growth and suggested the existence of an S6K1-specific substrate (or substrates) involved in this process.

S6K has been recently implicated in ageing. Since caloric restriction increases lifespan, Kapahi and co-workers investigated whether the nutrient sensing TOR-S6K pathway may be involved in the regulation of lifespan in Drosophila. Overexpression of the upstream inhibitor of TOR/S6K signalling, dTsc2, extended lifespan, as did overexpression of a dominant negative dTOR construct or kinase dead dS6K. Conversely, overexpression of a constitutively active dS6K mutant decreased lifespan (Kapahi et al. (2004) Curr. Biol. 14, 885-90). This study demonstrates that like other positive regulators of cell growth, dS6K plays a role in the aging process of Drosophila. A recent study using primary human endothelial cells has shown that PKB-mediated inhibition of FOXO3a is involved in the onset of senescence and that inhibition of PKB extends the lifespan of these cells in culture (Miyauchi et al. (2004) EMBO J. 23, 212-20). This suggests that the insulin signalling pathway is also important in regulating the lifespan of human cells. Interestingly, S6K1 was recently identified as a positive mediator of Ras-induced senescence, suggesting that it too may be a negative regulator of cellular lifespan in humans.

Deregulation of S6K function has been implicated with human pathologies, like cancer and diabetes. Mice lacking S6K1 display hypoinsulinaemia exhibit glucose intolerance (a phenotype closely resembling that of preclinical type II diabetes mellitus). Examination of the pancreatic beta cells of these mice revealed that they were significantly smaller than those of the wild type littermates and that therefore their capacity for insulin production was impaired (Pende et al. (2000) Nature 408, 994-997). This underlines the importance of S6K I-mediated signalling and demonstrates how defects in cell growth can have secondary effects on cellular function.

A recent study has also shown that S6K1^{-/-} mice are resistant to age- and diet-induced obesity. The rate of oxidative phosphorylation in white adipose tissue and muscle of S6K1^{-/-} mice is higher than in wild type animals. Accordingly, the mRNA levels of several genes involved in energy combustion and oxidative phosphorylation are highly upregulated in these tissues from the S6K1^{-/-} mice (Um et al. (2004) Nature 431, 200-205). As mentioned above, the hypoinsulinaemia and glucose intolerance manifest in the S6K1^{-/-} mice, would normally be expected to result in the development of insulin resistance seen in type II diabetes (Pende et al. (2000) Nature 408, 994-997). However, when mice were fed on high fat diets, the wild type animals developed insulin-resistance, while the S6K1^{-/-} mice remained insulin-sensitive. This was associated with a decrease in insulin signalling via PKB in wild type mice not seen in the knockouts (Um et al. (2004) Nature 431, 200-205). A major advance in linking S6K signalling to cancer has come from the study of two tumour suppressor genes, TSC1 and TSC2, which encode the proteins hamartin and tuberin, respectively (reviewed in Marygold and Leevers (2002) Curr. Biol. 12, R785-7; McManus and Alessi (2002) Nat. Cell. Biol. 214, E214-216). Patients suffering from the relatively common autosomal dominant disorder, tuberous sclerosis complex (TSC) all have mutations in either TSC1 or TSC2. Inheritance of germ line mutations or acquisition of sporadic mutations in these genes leads to the development of benign tumours (hamartomas) in multiple tissues, particularly in the kidneys, skin, heart, eyes and brain, in which they often cause mental retardation and seizures. Numerous observations have identified the Tsc1/2 heterodimer as the link between PI(3)K/PKB signalling and mTOR/S6K activation. They provide a mechanism by which growth factor signals and nutrient signals feed into a common downstream pathway and explain the high sensitivity of S6K activity to PI(3)K inhibitors such as wortmannin and LY-294002 (reviewed in Manning and Cantley, (2003) Trends Biochem. Sci. 28, 573-6). It has been clearly established that aberrant S6K signalling is a major feature of TSC (Goncharova et al. (2002) J. Cell. Biol. 167, 1171-82; Kenerson et al. (2002) Cancer Res. 62, 5645-50). However, the fact that TSC patients develop benign tumours confirms data suggesting that constitutive TOR-S6K signalling alone is not sufficient to induce complete cellular transformation. There is however, an accumulating body of evidence suggesting that constitutive S6K signalling is important to the growth of many tumours. A recent report shows a reduction in the level of tuberin in a number of sporadic squamous and basal cell carcinomas. This effect was shown to be epigenetic, suggesting that cancer cells that downregulate tuberin expression at the level of transcription or translation obtain a selective growth advantage over the surrounding normal cells (Wienecke et al. (2002) J. Cutan. Pathol. 29, 287-290).

The gene encoding S6K1 was found to be located to chromosomal region 17q23, which is a frequent site of gene amplification in breast cancer (Barlund et al. (2000) Cancer Res. 60, 5340-5344). The gene encoding S6K2 was mapped to chromosomal region 11q13, which is associated with acute myeloid leukemia, Non-Hodgkin's lymphoma, chronic lymphoblastic disorder. Furthermore, S6K1 amplifications were identified in both breast cancer tissues and cell lines. In addition, an elevated level of S6K1 expression have been demonstrated for other human malignancies, such as thyroid, endometrial and ovary cancer. S6K overexpression in human meningioma cell lines resulted in increased tumor size in vivo (Surace et al. (2004) Ann. Neurol. 56, 295-298). Taken together, these studies clearly indicate the involvement of S6K1 and 2 in malignant transformation.

So far, no direct and specific inhibitors for S6K have been identified. Under these circumstances, mTor inhibitor Rapamycin and PI3-K inhibitor Wortmannin have been used extensively to elucidate the function and regulation of S6Ks. These two unrelated inhibitors fully abrogate the activation of S6K induced by mitogenic stimuli. The development of water-soluble esters of rapamycin, such as CCI-779 and RAD001, which are potentially suitable for administration as anti-cancer agents has allowed such experiments to progress into clinical trials (reviewed in Hidalgo and Rowinsly (2000) Oncogene 19, 6680-6686; Bjornsti and Houghton (2004) Nat. Rev. Cancer 4, 335-348). In the USA, CCI-779 is currently being tested in thirteen phase II clinical trials for the treatment of a wide range of cancers and one phase III trial, for renal cell carcinoma (http://clinical trials.gov). These developments have encouraged further interest in the mTOR/S6K signalling pathway in the context of cancer. It is important to note, that Rapamycin derivatives demonstrate impressive activity against a broad range of human cancers and exhibit low toxicity (Hidalgo and Rowinsky (2000) Oncogene 19, 6680-6686).

Recent data from our laboratory implicate S6K not only in the regulation of gene expression at the level of translation, but also transcription. Using DNA microarrays and stable cells lines overexpressing activated forms of S6K1 and S6K2, we identified a panel of genes which are strongly induced by S6Ks. The mechanism of transcriptional activation by S6Ks is not known.

Gene expression in mammalian cells is achieved through the formation of multienzyme complexes around DNA sequences located in the regulatory regions of the gene, which is usually distinct from the coding region. The formation of regulatory complexes is facilitated by DNA binding proteins, such as transcription factors. DNA binding proteins possess binding domains which recognize and associate with DNA in sequence specific or sequence-independent manner (A. Klug, (1995) Ann. N.Y. Acad. Sci. 758, 143-160). A myriad of sequence specific DNA binding domains have been identified, including homeobox, zinc finger, leucine zipper, helix-loop-helix etc. DNA-binding HMG-box domains A and B or AT-hook motifs bind DNA without sequence specificity. However, HMG-box domains exhibit a high affinity for bent, distorted or bend linear DNA, while AT-hooks recognize DNA minor grove which is AT rich (Thomas (2001) Biochem. Soc. Trans, 29, 395-401).

The AT-hook is a small DNA-binding protein motif which was first described in the high mobility group non-histone chromosomal protein HMG-I(Y) (Reeves (2000) Environ. Health Perspect. 108, Suppl. 5, 803-809). Since its discovery, this motif has been observed in other DNA-binding proteins from a wide range of organisms. Unlike several of the other well-characterized DNA-binding motifs, the AT-hook is a small motif, which has a typical sequence pattern centered around a glycine-arginine-proline (GRP) tripeptide. The importance of this short conserved sequence is stressed by the observation that it is necessary and sufficient to bind DNA. In some proteins containing AT-hook motifs, there is a possibility that these may serve as accessory DNA-binding domains for several transcription factors, presumably to anchor them to particular DNA structures (e.g. minor grooves and four-way junctions). These AT-hook motifs seem to be auxiliary elements necessary for cooperation with other DNA-binding activities in the same or different proteins (Susbielle et al. (2005) Curr. Med. Chem. Anticancer Agents 5, 409-420; Reeves et al. (2003) J. Biol. Chem. 278, 42106-42114).

The AT-hook forms a C-shaped structure with the ‘rear’ of the concave surface inserted into the minor groove of the DNA. The proline residues in the AT-hook are probably responsible for maintenance of the rigid structure of this domain in the presence of DNA while the characteristic RGR sequence motif of the AT-hook adopts an extended structure and participates in DNA-protein interactions. The arginine residues are seen to penetrate deep into the B-DNA helix and interact with the bases of DNA.

Multiple or single AT-hook motifs are also found in several multi-domain proteins which associate with chromatin. Striking examples include the human HRX (ALL-1) protein, TAF_{II}250, Saccharomyces cerevisiae ASH1, S. cerevisiae SWI2, Drosophila melanogaster ISWI, ENBP1, M33 (vertebrate polycomb), doom/Mod(Mdg4) and tramtrack (Wassarman et al. (2001) J. Cell. Sci. 114, 2895-2902; Mohrmann et al. (2005) Biochim. Biophys. Acta. 1681, 59-73, Harvey et al. (1997) Mol Cell Biol. 17, 2835-2843). Several of these proteins are involved in the organization of chromatin into specific states. HRX (ALL-1) and ASH1 are members of the trithorax group of regulatory genes which are involved in chromatin decondensation. SWI2 and ISWI are ATP-dependent chromatin remodeling proteins. TAF_{II}250 is a basal transcription factor which is involved in transcription regulation and also contains histone acetyltransferase activity. In contrast, the POZ domain proteins Mod(Mdg4) and tramtrack act as regulators of transcriptional insulation and mediators of transcription repression, respectively.

The presence of AT-hooks in these proteins suggests that these chromatin architecture regulators probably use DNA sequence information in the form of AT-rich tracts, to target specific regions on the chromosomes. The AT-richness of SARs and their presumptive involvement in transcription activation suggest that the AT-hooks may indeed play a role in tethering these proteins to specific chromosomal regions. Both ARBP and its ortholog, the mammalian methylated DNA binding protein MeCP2, contain both an AT-hook motif and a methylated CpG DNA binding domain (Klose et al. (2005) Mol. Cell. 19, 667-78).

There are several examples where a single AT-hook motif was detected in proteins which have additional larger DNA-binding domains. In addition to the AT-hook D. melanogaster apterous, mammalian LH2 and Barx1 also contain homeodomains; human RFX5 protein also contains the DNA-binding RFX box; human ESE-1 protein also contains an ETS domain; S. cerevisiae SWI5, Drosophila castor and mouse CTCF1 also contain zinc fingers; the fungal metal binding transcription factors of the AMT1 family of proteins contain a zinc cluster. This suggests that in several cases, the AT-hook may serve as an additional peptide contact in the minor groove even as the principal or larger DNA-binding protein contacts the major groove. It is tempting to speculate that in these cases the AT-hook may serve as a ‘built-in’ cofactor like the HMG-I(Y) protein and subtly alter the affinity and specificity of the ‘principal or larger’ DNA-binding domain by recognizing sites in the vicinity of the principle DNA binding site. This may again be a general feature of eukaryotic DNA-binding proteins: small basic patches such as the N-terminus of homeodomains and the recently described GRIP box of nuclear hormone receptors, which contact the minor groove in addition to the main DNA-binding activity in the major groove.

In the protein kinase family, which is highly diverse and includes over 500 proteins, only few members were found to possess functional DNA binding domains. These are cABL tyrosine kinase, DNA-dependent protein kinase (DNA-PK), ATM kinase (Ataxia Telangiectasia Mutated). Numerous studies from various laboratories implicated these kinases in the regulation of transcription, genome stability, DNA repair, cell cycle control and survival (Taylor et al. (2005) J. Clin. Pathol. 58, 1009-1015; Meek et al. (2004) Immunol. Rev. 200, 132-141; Giles et al. (2005)). Deregulation of their function leads to cancer. Therefore, they are excellent targets for drug development (Hannah (2005) Curr. Mol. Med. 5, 625-42).

### SUMMARY OF THE INVENTION

The invention encompasses an isolated peptide or derivative thereof comprising a binding domain of p300 acetyltransferase of less than 200 amino acids of SEQ ID NO: 4 wherein the binding domain is capable of binding to and modulating an S6 kinase protein activity. In one embodiment, said binding domain binds to an activated form of S6 kinase protein and can comprise a histone acetyltransferase domain capable of acetylating the S6 kinase protein. One site of acetylation can be at an amino acid corresponding to residue 516 of an S6 kinase protein. In some embodiments, the acetylation site comprises a lysine residue.

The invention also includes isolated nucleic acid molecules encoding the above isolated peptides or derivatives thereof. In some embodiments, the isolated nucleic acid molecule specifically hybridizes under stringent conditions to a complementary nucleic acid molecule.

The invention also encompasses an isolated protein or fragments thereof comprising the A-T hook motif of S6 kinase 2. The isolated peptide may lack the ability to bind DNA. In one embodiment of the invention the isolate protein has one or more mutations, such as a substitution and/or deletion, at any amino acid residue corresponding to amino acids 487 to 495 of SEQ ID NO: 8. In another embodiment there is a substitution at amino acid residue 492 of SEQ ID NO: 8, which may be e.g. a substitution from Arginine to Proline.

The invention also includes nucleic acid molecules encoding such isolated proteins or fragments thereof. These nucleic acid molecules may be operably linked to one or more expression control elements. The nucleic acid molecules or fragments thereof may be part of a vector. Such vectors may be used to transform a host cell line which may consist of prokaryotic or eukaryotic cells.

The invention also encompasses methods of producing a polypeptide comprising culturing a host cell under conditions in which the polypeptide encoded by said nucleic acid molecule or fragment thereof is expressed.

The invention further encompasses methods of identifying an agent which binds to the S6 kinase protein, comprising exposing the S6 kinase protein to the agent in the presence of the isolated p300-4 peptide, and measuring the specific binding of the isolated peptide to the S6 kinase protein, wherein a decrease in the binding of the isolated peptide compared to a control is indicative of an agent capable of binding to the S6 kinase protein.

The invention also encompasses a method of identifying an agent which modulates the acetylation of the S6 kinase protein, comprising exposing the S6 kinase protein to the agent, and measuring the acetylation of the S6 kinase protein by the isolated peptide; wherein a change in the acetylation of S6 kinase compared to a control is indicative of an agent capable of modulating the acetylation of S6 kinase by the isolated peptide. In some embodiments of these methods, they further comprise contacting the S6 kinase protein with the above isolated peptide prior to measuring acetylation.

The invention also includes a method of identifying an agent which modulates an activity of the S6 kinase protein, comprising exposing the S6 kinase protein to the agent, measuring the acetylation at a lysine residue at position 516 of the S6 kinase protein by the agent, and measuring the activity of the S6 kinase protein, wherein a change in activity compared to a control is indicative of an agent capable of modulating an activity of the S6 kinase protein. In some embodiments of this method, the activity of the S6 kinase protein is determined by measuring phosphorylation of one or more additional proteins.

In further embodiments of all of these methods, the control includes a negative control. In one embodiment, the negative control does not comprise the agent. In all of these methods, a positive control can be included. In some embodiments, the positive control comprises a class I/II HDAC inhibitor such as Trichostatin-A.

The invention further encompasses methods of identifying an agent that modulates the binding of S6 kinases, preferably S6 kinase to a nucleic acid (e.g. DNA). In one embodiment, the method comprises exposing S6 kinase 2 protein to an agent; and detecting the binding of the S6 kinase 2 protein to the nucleic acid, wherein change in the level of binding compared to a control is indicative of an agent capable of modulating S6 kinase 2 binding to the nucleic acid. The method may further comprise assaying the activity of S6 kinase 2 by such means as e.g. phosphorylation of other proteins. The methods may utilize both positive and negative controls. In one embodiment, the positive control protein comprises the AT hook domain of S6K2 (corresponding to amino acids 487 to 495 of SEQ ID NO: 8). The invention also encompasses agents identified by such methods.

The invention also includes a method of modulating the activity of an S6 kinase protein comprising contacting the S6 kinase protein with the isolated peptide or derivative thereof as described herein. In some embodiments, the S6 kinase protein activity is inhibited. The invention further includes a method of treating a protein kinase related disease in a patient in need of such treatment comprising administering a therapeutically effective amount of a pharmaceutical composition comprising an agent capable of acetylating the protein kinase. In some embodiments, the agent is the isolated peptide described herein while in other embodiments the agent is a deacetylase inhibitor and/or a kinase inhibitor including S6 kinase inhibitors.

The invention further encompasses methods of identifying an agent that modulates the binding of S6 kinases, preferably S6 kinase to a nucleic acid (e.g. DNA). In one embodiment, the method comprises exposing S6 kinase 2 protein to an agent; and detecting the binding of the S6 kinase 2 protein to the nucleic acid, wherein change in the level of binding compared to a control is indicative of an agent capable of modulating S6 kinase 2 binding to the nucleic acid. The method may further comprise assaying the activity of S6 kinase 2 by such means as e.g. phosphorylation of other proteins. The methods may utilize both positive and negative controls. In one embodiment, the positive control protein comprises the AT hook domain of S6K2 (corresponding to amino acids 487 to 495 of SEQ ID NO: 8). The invention also encompasses agents identified by such methods.

The invention also includes methods of treating a disease associated with protein kinase (e.g. S6 kinase 2 protein) in a patient comprising administering to the patient of an effective amount of an agent to modulate the activity of the S6 kinase 2 protein. Such an agent may downregulate the activity of S6 kinase protein. Such an agent may be an antibody (e.g. a monoclonal antibody. In one embodiment, the S6 kinase 2 protein is human S6 kinase 2 (SEQ ID NO: 8). In another embodiment, the S6 kinase 2 protein has 95% homology to SEQ ID NO: 8.

Exemplary protein kinase related diseases include, but are not limited to, cancer, blood vessel proliferative disorders, autoimmune disorders, and metabolic diseases. Types of cancer which can be treated by the methods of the invention include, but are not limited to, squamous cell carcinoma, astrocytoma, Kaposi's sarcoma, glioblastoma, multiple myeloma, lung cancer, bladder cancer, head and neck cancer, melanoma, ovarian cancer, prostate cancer, breast cancer, small-cell lung cancer, glioma, colorectal cancer, genitourinary cancer, gastrointestinal cancer. Types of blood vessel proliferative disorders which can be treated by the methods of the invention include, but are not limited to, diabetic retinopathy, age-related macular degeneration, retinopathy of prematurity, arthritis and restenosis. Types of autoimmune disorders which can be treated by the methods of the invention include, but are not limited to, lupus erythematosus, discoid lupus erythematosus, subacute cutaneous lupus erythematosus, drug-induced lupus erythematosus, and systemic lupus erythematosus. Types of metabolic disorders which can be treated by the methods of the invention include, but are not limited to, psoriasis, diabetes mellitus, wound healing, inflammation and neurodegenerative diseases.

The invention also encompasses an antibody which specifically binds to an acetylation site on a kinase protein and is capable of inhibiting acetylation of the kinase protein by an acetyltransferase protein. In one embodiment, the antibody binds to S6 kinase on or near amino acid residue.

### BRIEF DESCRIPTION OF THE DRAWINGS

FIG. 1 shows binding of S6 kinases to acetyltransferases p300 and P/CAF. In FIG. 1A, S6K1 and S6K2 were immunoprecipitated from MCF-7 cells using polyclonal antibodies or pre-immune serum control. Co-immunoprecipitated p300 was detected by western blotting with specific antibody. In FIG. 1B, 293 cells were cotransfected with either EE-S6K1 and Gal4-p300 or empty vector and Gal4-p300. S6K1 was immunoprecipitated using anti-EE antibody and associated p300 was detected by western blotting with anti-Gal4 antibody. Input: approximately ten percent of soluble lysate. In FIG. 1C, NIH-3T3 cells were transfected with empty vector or EE-S6K2. Anti-EE immunoprecipitates were subjected to western blotting with anti-p300 antibody. Input: approximately one percent of soluble lysate. FIG. 1D demonstrates the interaction between S6 kinases with P/CAF in cells following serum stimulation. Endogenous S6K1 and S6K2 were immunoprecipitated from MCF-7 cells using polyclonal antibodies. Associated P/CAF was detected by western blotting with polyclonal antibodies (Upstate). In FIG. 1E, the full length p300 was used to produce several GST-fusion fragments as shown (Hasan et al. (2001) Mol. Cell. 7, 1221-1231). Expressed fusion proteins were coupled to glutathione beads and used in an in vitro GST pulldown assay with recombinant S6K2. After extensive washing, bound S6K2 was detected by western blotting.

FIG. 2 shows acetylation of S6 kinases by p300 and P/CAF in vitro and in vivo. In FIG. 2A, recombinant S6 kinases were acetylated in vitro using the purified acetyltransferase domain of p300 or full length PCAF and ¹⁴C-labelled acetyl CoA. Proteins were resolved by SDS-PAGE and acetylated proteins were analysed by phosphorimaging (Bio-rad). In FIG. 2B, recombinant S6K2 or GST were subject to in vitro acetylation reactions with p300 and 14C AcCoA. In vitro acetylation of other protein kinases is shown in FIG. 2C. Recombinant PKB/Akt, PKCa, PDK1 and PI3K (p85a/p110a) were purified using baculovirus expression system. In vitro acetylation assay was carried out as previously described. In FIG. 2D, HEK 293 cells were transfected with S6 kinases in the absence or presence of Gal4-p300 as shown. Cells were treated with medium containing either solvent control or TSA to a final concentration of 660 nM for twelve hours prior to harvesting. Recombinant S6 kinases were immunoprecipitated using the EE-antibody and acetylated S6 kinase was detected by western blotting with polyclonal acetylated lysine antibodies (Upstate). The level of immunoprecipitated S6K1 and S6K2 was analysed by reprobing the membranes with specific antibodies. The expression of Gal4-p300 was tested in total lysates using anti-Gal4 antibodies.

FIG. 3 shows identification of acetylation sites in S6K1 by mass spectrometry. In this study recombinant S6 kinases were acetylated in vitro using p300 immunoprecipitated from cells. Proteins were resolved by SDS-PAGE and S6 kinase bands from acetylation and control (no p300) reactions were excised after Coomassie staining. Proteins were subject to in-gel trypsin digest and MALDI-TOF mass spectrometry. An example of MS/MS analysis of an acetylated peptide in shown in FIG. 3A. The peptide shown was detected in the acetylated sample in the form of two peaks separated by 43 Da indicating presence of an acetyl group. The identified peptide (shown in the box) corresponds to the C-terminal region of S6K1, where K516 was found to be acetylated. FIG. 3B shows alignment of P-loop lysines in S6Ks from different species and FIG. 3C shows alignment with P-loops of other AGC kinases.

FIG. 4 shows substitution of S6K P-loop lysines to arginines or glutamines significantly inhibits S6 kinase activity. Wild type or mutant S6Ks were expressed in 293 cells, immunoprecipitated using anti-EE antibody and subjected to in vitro kinase assays. (A) Graphs showing the S6K activity of wild type S6K1 and S6K2 compared with constructs bearing substitutions of arginine (RR) or glutamine (QQ) for the P-loop lysines (P=0.1, n=3) (B) Representative autoradiograph of one such experiment. (C) Equal quantities of lysates from cells overexpressing wild type S6K1 or the P-loop mutants were blotted with anti-phospho-T412 or with anti-EE to compare S6K phosphorylation between wild type and mutant constructs.

FIG. 5 shows the position of the P-loop and conserved lysines in a putative model of the S6 kinase domain. The modelling of the S6 kinase domain was based on a crystal structure of Akt/PKB, a member of the AGC family. The model was generated using MODELLER7 programme with standard comparative modelling parameters. The location of two conserved lysines in the P-loop is indicated in red and blue. The rest of the loop is shown in green. Both lysines are well accessible on the surface of S6 kinase domain. The alignment of the P-loop sequences from various members of AGC family is shown.

FIG. 6 shows that acetylation affects S6K1 activity in cells. In FIG. 6A (upper panel), C2C12 myoblasts were treated for 18 hours with increasing concentrations of TSA or with the structurally unrelated HDAC inhibitor, sodium butyrate. Endogenous S6 kinase activity was assessed by western blotting against pT412 and against phosho-rpS6. In another experiment, C2C12 cells were treated with 1 μM TSA or control for the times shown and S6 kinase activity was measured by in vitro kinase assay using purified ribosomes as substrate (FIG. 6B). A representative autoradiograph is shown beneath the graph.

FIG. 7 shows the effect of histone deacetylase inhibitor TSA on S6K activity in a range of different cell types. (A) The cell lines indicated (see text) were treated with TSA and nicotinamide for eight hours. Following lysis, proteins were resolved by SDS-PAGE and western blots were probed as labelled to evaluate S6K activation. (B) MKOC cells (see text) were treated as in (A) and analysed by western blotting. S6K1 was also immunoprecipitated from lysates and subjected to immune complex kinase assay. (C) To check whether TSA or nicotinamide directly inhibit S6K1, recombinant purified S6K1 was used for kinase assays in the absence or presence of deacetylase inhibitors as indicated.

FIG. 8 shows the analysis of endogenous S6K1 acetylation during the cell cycle. NIH-3T3 cells were synchronized by serum starvation for twenty-four hours followed by addition of serum for the indicated times. Endogenous S6K1 was immunoprecipitated and acetylation was detected by western blotting with anti-acetyl lysine antibodies. Blots were reprobed with polyclonal antibodies against total S6K1. Lysates were resolved by SDS-PAGE and western blotting was carried out to measure S6K activation at each stage using anti-S6K pT412 and anti-pS6 (235/236) antibodies.

FIG. 9 shows testing the specificity of acetyl-Lysine specific S6K1 antibodies. Synthetic peptides, possessing acetylated lysines identified by mass spectrometry, were used for the immunisation of rabbits. Generated antibodies were initially purified on a column containing acetylated peptide. Then, purified antibodies were loaded on a column containing corresponding non-acetylated peptides in order to remove IgGs which recognise non-acetylated form of S6K. The specificity of purified antibodies was tested by Western blotting of total lysates or immunoprecipitates from Hek293 cells transfected with wtS6K1, K516R S6K mutant and vector alone.

FIG. 10 shows S6K1 is acetylated at K516 in vivo. FIG. 10A shows that acetylation of S6K1 at K516 is induced by serum stimulation. In this study, NIN3T3 cells were starved and stimulated with serum for various period of time. Total cell lysates were separated by SDS-PAGE and immunoblotted with acetyl-K516 antibodies. In FIG. 10B, the effect of various cell signalling inhibitors on K516 acetylation is shown. In this study, NIH3T3 cells were starved and treated with 50 nM Rapamycin (mTor inhibitor), 20 μM LY294002 (phosphatidylinositol 3′-kinase inhibitor), 10 μM PD98059 (MAP kinase inhibitor) or vehicle alone thirty minutes before stimulation with serum. Then, cells were stimulated with serum for one hour and lysed. The supernatants of total lysates were separated by SDS-PAGE and Western blotted with acetyl-K516 antibodies. FIG. 10C shows that acetylation of S6K1 at K516 does not affect its kinase activity. The wtS6K and K516R mutants were transiently expressed in Hek293 cells. Immunoprecipitated recombinant S6Ks and total cell lysates were resolved by gel electrophoresis and immunoblotted with antibodies specific for EE tag, pT412 and actin.

FIG. 11 shows S6K2CT binding to DNA in vitro. S6K1 and S6K2 C-terminal domains were coupled to nickel-NTA beads and assayed for binding to ³²P labeled genomic DNA. Bound DNA was measured by scintillation counting of beads following extensive washing.

FIG. 12 shows the in vitro DNA binding of S6K2 and S6K1. FIG. 12A shows that full length S6K2 but not S6K1 binds DNA in vitro. Recombinant EE-S6K1 and EE-S6K2 were immobilized on protein-G sepharose using anti-EE antibody and assayed for binding to ³²P labeled genomic DNA. Bound DNA was measured by scintillation counting of beads following extensive washing. FIG. 12B shows an electrophoretic mobility shift assay mobility shift assay (EMSA), thus demonstrating binding of recombinant EE-S6K2 to genomic DNA in vitro.

FIG. 13 shows that the S6K2 C-terminal domain is required for DNA binding. FIG. 3A shows the in vitro binding of DNA to the C-terminal domain of S6K2. HEK cells were transiently transfected with EE-S6K1, EE-S6K2 or with EE-S6K2ΔCT. Proteins were then immunoprecipitated using anti-EE and used in an in vitro DNA binding assay with genomic DNA. FIG. 13B shows expression of the recombinant protein. Total protein (30 μg) from soluble lysates were subjected to western blotting with anti-EE to measure expression of the recombinant proteins.

FIG. 14 shows that mutation of the S6K2 AT-hook motif inhibits binding of the full length protein to DNA. FIG. 14A compares the DNA binding abilities of S6K2T WT and R492P. S6K2 WT and R492P were overexpressed in 293 cells and immunoprecipitated using anti-EE antibody. DNA binding to genomic DNA was assayed. FIG. 14B shows the expression of the recombinant proteins. 30 μg of total protein from soluble lysates were subjected to western blotting with anti-EE to measure expression of the recombinant proteins.

FIG. 15 shows the effect of DNA binding on S6K2 kinase activity. FIG. 15A is an analysis of S6K2 kinase assay in vitro in the presence of increasing concentration of DNA. FIG. 15B is a bar chart representing the results from the in vitro kinase assay in 15A. DNA-induced stimulation of S6K2 activity was observed in at least four different experiments.

### DETAILED DESCRIPTION

The present invention is based on the discovery that, in addition to phosphorylation, protein kinases can also be modulated by acetylation. This is the first time that any kinase has been shown to be modulated by acetylation. The present invention is based upon the finding that S6 kinase protein is acetylated both in vivo and in vitro by p300 and P/CAF acetyltransferases. More accurate to say that in conditions under which S6K acetylation is increased (i.e. presence of HDAC inhibitors/overexpression of p300), S6 kinase activity and 412 phosphorylation are reduced. Mutation of the P-loop lysines (which are acetylated by p300 in vitro) to glutamine, which mimics acetylation, results in the complete inactivation of S6Ks and loss of 412 phosphorylation. The present invention is also based on the discovery that S6K2 possesses an AT-hook DNA binding motif. This is also the first time that any protein kinase has been shown to contain a functional AT-hook DNA binding motif. The present invention is based upon the finding that S6 kinase 2 protein binds to DNA and is thereby activated which in turn stimulates its kinase activity. Based upon these data a novel mechanism is proposed by which S6K2 can transduce growth-promoting effects in response to mitogens and nutrients. This may involve the regulation of transcription factors and/or chromatin remodeling proteins by phosphorylation, when S6K2 complexes with DNA and is activated by this interaction. This discovery has important implications for the development of drugs which can block S6K2-DNA interaction and subsequently its growth promoting functions, which are deregulated in cancer and diabetes.

It has been determined that this active domain of p300 acetyltransferase (p300-4) extends from amino acid 1,459 to 1,892 of this protein (SEQ ID NO: 6). Thus, any agent which modulates the acetylation of a protein kinase, including but not limited to the p300-4 fragment and its derivatives, can be used for the treatment of cell proliferative disorders associated with aberrant protein kinase activity.

The present invention also encompasses methods of modulating acetylation of a protein kinase utilizing acetyltransferase binding domains from other proteins in the histone acetyltransferase family of proteins which are similar to p300 acetyltransferase. In one embodiment, the acetyltransferase binding domain is from CBP (CREB-binding protein), which is a highly homologous to p300 acetyltransferase.

### DEFINITIONS

As used herein, the term “p300 acetyltransferase binding domain” refers to a region of the p300 acetyltransferase (SEQ ID NO: 4) which interacts or binds to one or more species of S6 kinase protein including human. The interaction or binding may be by one or more of a variety of mechanisms including, but not limited to ionic interactions, covalent interactions, hydrophobic interactions, van der Waals interactions, etc. In one embodiment, the binding domain includes the fragment of p300 acetyltransferase known as p300-4 (i.e., p300-4 fragment, SEQ ID NO: 6). In another embodiment, the binding domain contains a histone acetyltransferase domain. The terms “p300 acetyltransferase binding domain” or “binding domain” are synonymous and used interchangeably throughout the specification. As used herein, “p300 acetyltransferase binding domain” does not include the full-length p300 acetyltransferase protein.

As used herein, the term “p300 acetyltransferase binding domain polypeptide” refers to any polypeptide containing a p300 acetyltransferase binding domain including fusion proteins, chimeras, variants, etc. In one embodiment, the p300 acetyltransferase binding domain polypeptide is capable of binding to a S6 kinase, including human S6 kinase. In a further embodiment, the p300 acetyltransferase binding domain polypeptide is capable of acetylating an S6 kinase, including human S6 kinase.

As used herein, the term “acetyltransferase” is used to refer to an enzyme responsible for the covalent modification of proteins via acetylation. In one embodiment, the acetyltransferase is a p300 acetyltransferase as set forth in SEQ ID NO: 4, a family member of the p300/CBP family of co-activators which have histone acetyltransferase activity. For the purposes of the present invention, reference to p300/CBP refers to human allelic and synthetic variants of p300 acetyltransferase, and to other mammalian variants and allelic and synthetic variants thereof, as well as fragments of said human and mammalian forms of p300 acetyltransferase. The p300 acetyltransferase molecule will however retain the ability to physically associate both in vitro and in vivo with S6 kinase protein. For the purposes of the present invention, the precise form and structure of a p300-acetyltransferase protein or fragment thereof may be varied by those of skill in the art, having regard to the particular assay format to be used.

As used herein, the term “S6 kinase protein” refers to p70 S6 kinase protein that is capable of transferring a phosphate group from ATP to an acceptor group of protein. It also refers to the full sequence of the protein or any suitable derivative or fragment thereof or fusion of S6 kinase protein or derivative, or fragment thereof. Examples include, but are not limited to, subtypes of this protein such as S6K1 and S6K2.

As used herein, the term “S6K2 derivative” refers to a S6K2 protein comprising one or more amino acid mutations in the AT-hook domain to prevent DNA binding. The mutations may be substitutions and/or deletions.

As used herein, the term “AT-hook motif” refers to an amino acid sequence, which contains a short DNA binding motif capable of binding to the minor groove of short (4 to 6 nucleotide base pairs) ‘AT-rich’ (adenine-thymine) DNA. Examples of AT-hook motifs, include, but are not limited to the amino acid sequences set forth in Table 2. As used herein, the term “AT-hook motif” is used interchangeably with the term “AT-hook domain” throughout the specification.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, and materials are described.

Polypeptides Containing p300 Acetyltransferase Binding Domain

As used herein, the term “p300 acetyltransferase binding domain derivative” refers to derivatives, analogs, variants, polypeptide fragments and mimetics of the p300 acetyltransferase binding domain and related peptides which retain the same activity as the p300-4 fragment, such as binding specifically to S6 kinase protein. Such p300 acetyltransferase binding domain derivatives can also be used for practicing the methods of the invention. Examples of derivatives include, but are not limited to, peptide variants of p300 acetyltransferase binding domain, for example, fragments comprising or consisting of contiguous n-mer peptides of SEQ ID NO: 4 or comprising at least about amino acid residues 50 to 100 or 150 to 200 of SEQ ID NO: 4, core binding sequences, and peptide mimetics.

Peptides containing a p300 acetyltransferase binding domain and derivatives thereof can be prepared using standard solid phase (or solution phase) peptide synthesis methods, as is known in the art. In addition, the nucleic acids encoding these peptides may be synthesized using commercially available oligonucleotide synthesis instrumentation and produced recombinantly using standard recombinant production systems. Production using solid phase peptide synthesis is necessitated if non-gene-encoded amino acids are to be included. The term “p300 acetyltransferase binding domain derivative” as used herein is synonymous with “variant” and also includes modifications to the p300 acetyltransferase binding domain by one or more deletions of up to ten (e.g., 1 to 2 or 1 to 5) amino acids; insertions of a total of up to ten (e.g., 1 to 5) amino acids internally within the amino acid sequence of p300-4 fragment; or up to a total of 100 amino acids at either terminus of the p300 acetyltransferase binding domain sequence; or conservative substitutions of a total of up to fifteen (e.g., 1 to 5) amino acids.

Derivatives of p300 acetyltransferase binding domain include polypeptides comprising a conservative or non-conservative substitution of at least one amino acid residue when the derivative sequence and the p300 acetyltransferase binding domain sequence are maximally aligned. The substitution may be one which enhances at least one property or function of the p300 acetyltransferase binding domain, inhibits at least one property or function of the p300 acetyltransferase binding domain, or is neutral to at least one property or function of the p300 acetyltransferase binding domain. As used herein, a “property or function” of the p300 acetyltransferase binding domain includes, but is not limited to, at least one selected from the group consisting of the ability to inhibit S6 kinase protein activity, arrest abnormal cell growth, specific binding to a benign or malignant cancer cell when compared to a non-cancer cell (i.e., normal), and killing of a benign or malignant cancer cell. In terms of the present disclosure, the cancer cell may be in vivo, ex vivo, in vitro, a primary isolate from a subject, a cultured cell, or a cell line.

Peptide variants include, but are not limited to, deletion or conservative amino acid substitution variants of SEQ ID NO: 6. As used herein, a conservative variant refers to alterations in the amino acid sequence that do not adversely substantially affect the biological functions of the peptide. A substitution, insertion, or deletion is said to adversely affect the peptide when the altered sequence substantially prevents or disrupts a biological function associated with the peptide (e.g., acetylation). For example, the overall charge, structure, or hydrophobic/hydrophilic properties of the peptide can be altered without adversely affecting a biological activity. Accordingly, the amino acid sequence can be altered, for example to render the peptide more hydrophobic or hydrophilic, without adversely affecting the biological activities of the peptide.

As discussed above, various changes can often be made to the amino acid sequence of a polypeptide which has a desired property in order to produce variants which still have that property. Such variants of the polypeptides are within the scope of the present invention. An example of a variant of the present invention is a polypeptide with the conservative substitution of one or more amino acids with one or more other amino acids. One skilled in the art is aware that various amino acids have similar characteristics. One or more such amino acids of a polypeptide can often be substituted by one or more other such amino acids without eliminating a desired property of that polypeptide (such as acetyltransferase activity). For example, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids that can often be substituted for one another include phenylalanine, tyrosine and tryptophan (amino acids having aromatic side chains), lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains).

Deletion and insertion variants of the polypeptides are within the scope of the present invention. Amino acid deletions can be advantageous since the overall length and the molecular weight of a polypeptide can be reduced whilst still retaining a desired activity. This can enable the amount of polypeptide required for a particular purpose to be reduced. Amino acid insertions relative to a polypeptide can also be made. This may be done to alter the nature of the polypeptide (e.g., to assist in identification, purification or expression). Polypeptides incorporating amino acid changes (whether substitutions, deletions or insertions) relative to the sequence of a polypeptide as defined above can be provided using any suitable techniques. For example, a nucleic acid sequence incorporating a desired sequence change can be provided by site-directed mutagenesis. This can then be used to allow the expression of a polypeptide having a corresponding change in its amino acid sequence.

The methods of the invention include corresponding polypeptides that display similar or related activity to p300-4 fragment for the diagnosis and treatment of diseases associated S6 kinase expression and activity including those with abnormal cell proliferation as described herein, including cancer. For purposes of the specification, “similar or related activity” is defined as binding to S6 kinase protein expressed by cells displaying abnormal cell growth, including benign cells exhibiting abnormal growth and malignant cancer cells. Examples of such polypeptide include, but are not limited to, polypeptides which contain one or more of the binding domains set forth in SEQ ID NO: 6.

Homology or sequence identity at the nucleotide or amino acid sequence level is determined by BLAST (Basic Local Alignment Search Tool) analysis using the algorithm employed by the programs blastp, blastn, blastx, tblastn and tblastx (Altschul et al. (1997) Nucleic Acids Res. 25, 3389-3402 and Karlin et al. (1990) Proc. Natl. Acad. Sci. USA 87, 2264-2268, both fully incorporated by reference) which are tailored for sequence similarity searching. The approach used by the BLAST program is to first consider similar segments, with gaps (non-contiguous) and without gaps (contiguous), between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994) Nature Genetics 6, 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al. (1992) Proc. Natl. Acad. Sci. USA 89, 10915-10919, fully incorporated by reference), recommended for query sequences over eighty-five nucleotides or amino acids in length.

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The present invention encompasses allelic variants, conservative substitution variants, and members of the p300 acetyltransferase binding domain having an amino acid sequence identity or homology of at least about seventy-five percent, at least about eighty-five percent, at least about ninety percent sequence, at least about ninety-five percent, at least about ninety-nine percent sequence identity with the entire p300-4 sequence set forth in SEQ ID NO: 6. As used herein, the terms “sequence identity” and “sequence homology” are synonymous. Identity or homology with respect to such sequences is defined herein as the percentage of amino acid residues in the candidate sequence that are identical with the known peptides, after alignment the sequences.

The polypeptide variants include peptides having a fragment of the amino acid sequence set forth in SEQ ID NO: 4, having at least about 10, 20, 30, 40, 50, 100, 200, 300, or 400 contiguous amino acid residues. The polypeptide variants further include those fragments associated with the activity of containing the p300 acetyltransferase binding domain or fragments thereof. Such fragments may contain functional regions of the p300 acetyltransferase binding as well as regions of pronounced hydrophilicity. Variants may also include a polypeptide containing the p300 acetyltransferase binding domain or fragments thereof linked to a second polypeptide, in any order, with intervening amino acids removed or replaced by a linker sequence.

### S6K2 Protein Derivatives

The present invention provides isolated proteins, allelic variants of the proteins, and conservative amino acid substitutions of the protein comprising the amino acids sequence of SEQ ID NO: 8 which contain one or more mutations at amino acids corresponding to residues 487 to 495. As used herein, the “protein” or “polypeptide” refers, in part, to a protein that has the human amino acid sequence depicted in SEQ ID NO: 8 and contains any of the aforementioned mutations. The terms also refer to proteins that have a slightly different amino acid sequence than that specifically recited above.

The proteins of the present invention are preferably in isolated form. As used herein, a protein is said to be isolated when physical, mechanical, or chemical methods are employed to remove the protein from cellular constituents that are normally associated with the protein. A skilled artisan can readily employ standard purification methods to obtain an isolated protein.

The proteins of the present invention further include insertion, deletion, or conservative amino acid substitution variants of SEQ ID NO: 8 including those defined herein at amino acids corresponding to residues 487 to 495. As used herein, a conservative variant refers to alterations in the amino acid sequence that does not adversely affect the biological functions of the protein. Here conservative variants would retain their ability to be acetylated, the ability to phosphorylate second proteins and the ability to bind DNA. A substitution, insertion, or deletion is said to adversely affect the protein when the altered sequence prevents or disrupts a biological function associated with the protein. For example, the overall charge, structure, or hydrophobic/hydrophilic properties of the protein can be altered without adversely affecting a biological activity. Accordingly, the amino acid sequence can be altered, for example to render the peptide more hydrophobic or hydrophilic, without adversely affecting the biological activities of the protein.

Ordinarily, the conservative substitution variants, and the members of the protein family, will have an amino acid sequence having at least about 75% amino acid sequence identity with the entire sequence set forth in SEQ ID NO: 8, more preferably at least about 80%, even more preferably at least about 90%, and most preferably at least about 95% sequence identity. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. Fusion proteins, or N-terminal, C-terminal or internal extensions, deletions, or insertions into the peptide sequence shall not be construed as affecting homology.

Thus, the proteins of the present invention include molecules having the amino acid sequence disclosed in SEQ ID NO: 8 and amino acid sequence variants wherein one or more amino acid residues has been inserted N- or C-terminal to, or within, the disclosed coding sequence; and amino acid sequence variants of the disclosed sequence, or their fragments as defined above, that have been substituted by at least one residue. Such fragments, also referred to as peptides or polypeptides, may contain functional regions of the protein identified as regions of the amino acid sequence which correspond to known protein domains, such as the AT-hook domain, as well as regions of pronounced hydrophilicity. The regions are all easily identifiable by using commonly available protein sequence analysis software such as MacVector (Oxford Molecular).

The present invention further provides compositions comprising a protein or polypeptide of the invention and a diluent. Suitable diluents can be aqueous or non-aqueous solvents or a combination thereof, and can comprise additional components, for example water-soluble salts or glycerol, that contribute to the stability, solubility, activity, and/or storage of the protein or polypeptide.

As described below, members of the family of S6K2 proteins can be used: (1) to identify agents which modulate the level of expression or at least one activity of the protein, (2) to identify binding partners for the protein, and (3) as a therapeutic agent or target.

Contemplated variants of polypeptides containing p300 acetyltransferase binding domain and/or of S6K2 protein derivatives further include those containing predetermined mutations by, e.g., homologous recombination, site-directed or PCR mutagenesis, and the corresponding proteins of other animal species, including but not limited to rabbit, mouse, rat, porcine, bovine, ovine, equine and non-human primate species, and the alleles or other naturally occurring variants of the family of proteins; and derivatives wherein the protein has been covalently modified by substitution, chemical, enzymatic, or other appropriate means with a moiety other than a naturally occurring amino acid (for example a detectable moiety such as an enzyme or radioisotope).

### Nucleic Acids Molecules

The present invention further provides nucleic acid molecules that encode the p300 acetyltransferase binding domain and derivative polypeptides of the invention. The present invention also provides nucleic acids that encode proteins comprising one or more mutations at any amino acid residue corresponding to amino acids 487 to 495 of SEQ ID NO: 8 and derivative polypeptides of the invention In one embodiment, these nucleic acids encode a p300 acetyltransferase binding domain polypeptide capable of binding to S6 kinase, including human S6 kinase. In another embodiment, these nucleic acids encode a p300 acetyltransferase binding domain polypeptide capable of acetylating a S6 kinase, including human S6 kinase. As used herein, “nucleic acid” is defined as RNA or DNA that encodes a protein or peptide as defined above, is complementary to a nucleic acid sequence encoding such peptides, hybridizes to a nucleic acid encoding a p300 acetyltransferase binding domain or to amino acids 487 to 495 of SEQ ID NO: 8 across the open reading frame under appropriate stringency conditions, or encodes a polypeptide that shares at least about 75% sequence identity, preferably at least about 80%, more preferably at least about 85%, and even more preferably at least about 90% or even 95% or more identity with the entire contiguous amino acid sequence of SEQ ID NO: 6 or 8. The “nucleic acid” of the invention further includes nucleic acid molecules that share at least 80%, preferably at least about 85%, and more preferably at least about 90% or 95% or more identity with the nucleotide sequence of SEQ ID NO: 5 or 7 particularly across the open reading frame. Specifically contemplated are genomic DNA, cDNA, mRNA and antisense molecules, as well as nucleic acids based on alternative backbones or including alternative bases whether derived from natural sources or synthesized. Such nucleic acids, however, are defined further as being novel and unobvious over any prior art nucleic acid including that which encodes, hybridizes under appropriate stringency conditions, or is complementary to nucleic acid encoding a protein according to the present invention.

“Stringent conditions” are those that (1) employ low ionic strength and high temperature for washing, for example, 0.015 M NaCl/0.0015 M sodium citrate/0.1% SDS at a temperature of at least 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer (pH 6.5) with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at a temperature of at least 42° C., with washes at 42° C. in 0.2×SSC and 0.1% SDS. A skilled artisan can readily determine and vary the stringency conditions appropriately to obtain a clear and detectable hybridization signal. Preferred molecules are those that hybridize under the above conditions to the complement of SEQ ID NO: 5 or 7 and which encode a functional protein. Even more preferred hybridizing molecules are those that hybridize under the above conditions to the complement strand of the open reading frame of SEQ ID NO: 5 or 7.

As used herein, a nucleic acid molecule is said to be “isolated” when the nucleic acid molecule is substantially separated from contaminant nucleic acid molecules encoding other polypeptides.

The present invention further provides fragments of the encoding nucleic acid molecule. As used herein, a fragment of an encoding nucleic acid molecule refers to a small portion of the entire protein coding sequence. The size of the fragment will be determined by the intended use. For example, if the fragment is chosen so as to encode an active portion of the protein, the fragment will need to be large enough to encode the functional regions of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming.

Fragments of the encoding nucleic acid molecules of the present invention (i.e., synthetic oligonucleotides) that are used as probes or specific primers for the polymerase chain reaction (PCR), or to synthesize gene sequences encoding proteins of the invention, can easily be synthesized by chemical techniques, for example, the phosphotriester method of Matteucci et al. (1981) J. Am. Chem. Soc. 103, 3185-3191 or using automated synthesis methods. In addition, larger DNA segments can readily be prepared by well known methods, such as synthesis of a group of oligonucleotides that define various modular segments of the gene, followed by ligation of oligonucleotides to build the complete modified gene. In a preferred embodiment, the nucleic acid molecule of the present invention contains a contiguous open reading frame of at least about three-thousand and forty-five nucleotides.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention. Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

As used herein, a “nucleic acid” refers to the polymeric form of ribonucleotide or deoxyribonucleotides (adenine, guanine, thymine, and/or cytosine) in either its single stranded form, or in double-stranded helix. This term refers only to the primary and secondary structure of the molecule and is not limited to any particular tertiary form. Thus, this term includes single-stranded RNA or DNA, double-stranded DNA found in linear DNA molecules (e.g., restriction fragments), viruses, plasmids, and chromosomes. Sequences of double-stranded DNA molecules, sequences may be described herein according to the normal convention of giving only the sequence in the 5′ to 3′ direction along the non-transcribed strand of DNA (e.g., the strand having a sequence homologous to the mRNA).

A nucleic acid “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

As used herein, a “promoter sequence” is a DNA regulatory region capable of binding RNA polymerase in a cell and initiating transcription of a downstream (3′ direction) coding sequence. For purposes of defining the present invention, the promoter sequence is bounded (inclusively) at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes.

A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide that directs the polypeptide to the cell surface or instructs the host cell to secrete the polypeptide to the extracellular space. This signal peptide is clipped off by the host cell before the protein leaves the cell. Signal sequences can be found associated with a variety of proteins native to prokaryotes and eukaryotes. For instance, alpha-factor, a native yeast protein, is secreted from yeast, and its signal sequence can be attached to heterologous proteins to be secreted into the media (see U.S. Pat. No. 4,546,082). Further, the alpha-factor and its analogs have been found to secrete heterologous proteins from a variety of yeast, such as Saccharomyces and Kluyveromyces (EP 88312306.9, EP 0324274, and EP 0301669). An example for use in mammalian cells is the tPA signal used for expressing Factor VIIc light chain.

A cell has been “transformed” by a exogenous or heterologous nucleic acid when such nucleic acid as been introduced inside the cell. The transforming nucleic acid may or may not be integrated (covalently linked) into chromosomal DNA making up the genome of the cell. In prokaryotes, for example, the transforming nucleic acid may be maintained on an episomal element such as a plasmid or viral vector. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming nucleic acid.

As used herein, a “cell line” is a clone of a primary cell that is capable of stable growth in vitro for many generations. As used herein, nucleic acid sequences display “substantial identity” when at least about 85 percent (preferably at least about 90 percent and most preferably at least about 95 percent) of the nucleotides match over the defined length of the nucleotide sequences. Sequences that are substantially identical can be identified in a Southern hybridization experiment under, for example, stringent conditions as defined for that particular system. Defining appropriate hybridization conditions is within the skill of the art.

A “heterologous” region of the nucleic acid construct is an identifiable segment of a nucleic acid within a larger nucleic acid molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene).

Vectors are used to simplify manipulation of the nucleic acids, which encode proteins of the invention, either for preparation of large quantities of nucleic acids for further processing (cloning vectors) or for expression of the polypeptides (expression vectors). Such proteins include the p300-4 fragment, proteins comprising one or more mutations at any amino acid residue corresponding to amino acids 487 to 495 of SEQ ID NO: 8 and derivative polypeptides, thereof. Vectors comprise plasmids, viruses (including phage), and integrated DNA fragments (i.e., fragments that are integrated into the host genome by recombination). Cloning vectors need not contain expression control sequences. However, control sequences in an expression vector include transcriptional and translational control sequences such as a transcriptional promoter, a sequence encoding suitable ribosome binding sites, and sequences, which control termination of transcription and translation. In one embodiment, the expression vector should preferably include a selection gene to facilitate the stable expression of the p300-4 fragment gene and/or to identify transformed cells. In another embodiment, the expression vector should preferably include a selection gene to facilitate the stable expression of the S6K2 fragment gene and/or to identify transformed cells. However, the selection gene for maintaining expression can be supplied by a separate vector in co-transformation systems using eukaryotic host cells.

Suitable vectors generally will contain a replicon (origins of replication, for use in non-integrative vectors) and control sequences which are derived from species compatible with the intended expression host. By the term “replicable” vector as used herein, it is intended to encompass vectors containing such replicons as well as vectors which are replicated by integration into the host genome. Transformed host cells are cells which have been transformed or transfected either with vectors containing p300-4 fragment or p300-4 fragment derivative polypeptide encoding nucleic acid or with vectors containing S6K2 fragment or S6K2 fragment derivative polypeptide encoding nucleic acid. The expressed polypeptides may be secreted into the culture supernatant, under the control of suitable processing signals in the expressed peptide (e.g. homologous or heterologous signal sequences).

Expression vectors for host cells ordinarily include an origin of replication, a promoter located upstream from the p300 acetyltransferase binding domain polypeptide coding sequence or the S6K2 coding sequence, together with a ribosome binding site, a polyadenylation site, and a transcriptional termination sequence. Those of ordinary skill will appreciate that certain of these sequences are not required for expression in certain hosts. An expression vector for use with microbes need only contain an origin of replication recognized by the host, a promoter which will function in the host, and a selection gene.

Commonly used promoters are derived from polyoma, bovine papilloma virus, CMV (cytomegalovirus, either murine or human), Rouse sarcoma virus, adenovirus, and simian virus 40 (SV40). Other control sequences (e.g., terminator, polyA, enhancer, or amplification sequences) can also be used.

An expression vector is constructed so that the p300-4 fragment or S6K2 protein or derivative polypeptide coding sequence is located in the vector with the appropriate regulatory sequences. The positioning and orientation of the coding sequence with respect to the control sequences is such that the coding sequence is transcribed and translated under the “control” of the control sequences i.e., RNA polymerase which binds to the DNA molecule at the control sequences transcribes the coding sequence. The control sequences may be ligated to the coding sequence prior to insertion into a vector, such as the cloning vectors described above. Alternatively, the coding sequence can be cloned directly into an expression vector which already contains the control sequences and an appropriate restriction site. If the selected host cell is a mammalian cell, the control sequences can be heterologous or homologous to the p300-4 fragment, S6K2 protein or derivative polypeptide coding sequence, and the coding sequence can either be genomic DNA containing introns or cDNA.

Higher eukaryotic cell cultures may be used to express the proteins of the present invention, whether from vertebrate or invertebrate cells, including insects, and the procedures of propagation thereof are known.

Other expression vectors are those for use in eukaryotic systems. An exemplary eukaryotic expression system is that employing vaccinia virus, which is well-known in the art (see, for example, WO 86/07593). Yeast expression vectors are known in the art (see, for example, U.S. Pat. Nos. 4,446,235 and 4,430,428). Another expression system is vector pHSI, which transforms Chinese hamster ovary cells (see WO 87/02062). Mammalian tissue may be cotransformed with DNA encoding a selectable marker such as dihydrofolate reductase (DHFR) or thymidine kinase and DNA encoding the p300-4 fragment or p300-4 fragment derivative polypeptide or S6K2 protein or S6K2 derivative peptide. If wild type DHFR gene is employed, it is preferable to select a host cell which is deficient in DHFR, thus permitting the use of the DHFR coding sequence as marker for successful transfection in hgt medium, which lacks hypoxanthine, glycine, and thymidine.

Depending on the expression system and host selected, the peptide of interest, such as p300 acetyltransferase binding domain polypeptides and S6K2 protein, are produced by growing host cells transformed by an exogenous or heterologous DNA construct, such as an expression vector described above under conditions whereby the polypeptide is expressed. The peptide of interest, such as p300-4 fragment, S6K2 protein or derivative polypeptides thereof, is then isolated from the host cells and purified. If the expression system secretes the protein or peptide into the growth media, the protein can be purified directly from cell-free media. The selection of the appropriate growth conditions and initial crude recovery methods are within the skill of the art.

Once a coding sequence for a p300 acetyltransferase binding domain polypeptide or S6K2 protein of the invention has been prepared or isolated, it can be cloned into any suitable vector and thereby maintained in a composition of cells which is substantially free of cells that do not contain any p300-4 fragment or S6K2 protein coding sequence, respectively. As described above, numerous cloning vectors are known to those of skill in the art.

### Binding Domain Peptide Mimetics

The present invention includes another class of p300 acetyltransferase binding domain derivatives called peptide mimetics that mimic the three-dimensional structure of p300-4 fragment. The present invention also includes peptide mimetics that mimic the three-dimensional structure of S6K2. Such peptide mimetics may have significant advantages over naturally occurring peptides including, for example, more economical production, greater chemical stability, enhanced pharmacological properties (half-life, absorption, potency, efficacy, etc.), altered specificity (e.g., a broad-spectrum of biological activities), reduced antigenicity and others.

In one form, mimetics are peptide-containing molecules that mimic elements of p300 acetyltransferase binding domain secondary structure. In another embodiment, the mimetics are capable of binding to S6 kinase, including human S6 kinase. In a further embodiment, the mimetics are capable of modulating the activity of the S6 kinase by effecting acetylation at the binding domain. In one embodiment, the mimetics facilitate acetylation while in another embodiment, they inhibit acetylation by another acetyltransferase (e.g., p300).

In an alternate form, mimetics are peptide-containing molecules that mimic elements of S6K2 AT-hook motif secondary structure. In yet another embodiment, the mimetics are capable of binding to S6K2, including human S6K2. In an alternate embodiment, the mimetics are capable of modulating the activity of the S6K2 by effecting DNA binding.

The underlying rationale behind the use of peptide mimetics is that the peptide backbone of proteins exists chiefly to orient amino acid side chains in such a way as to facilitate molecular interactions, similar to those of antibody and antigen. A peptide mimetic is expected to permit molecular interactions similar to the natural molecule. In another form, peptide analogs are commonly produced in the pharmaceutical industry as non-peptide drugs with properties analogous to those of the template peptide. These types of non-peptide compounds are also referred to as peptide mimetics or peptidomimetics (Fauchere (1986) Adv. Drug Res. 15, 29-69; Veber & Freidinger (1985) Trends Neurosci. 8, 392-396; Evans et al. (1987) J. Med. Chem. 30, 1229-1239 which are incorporated herein by reference) and are usually developed with the aid of computerized molecular modeling.

Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a chemical linkage not normally found in peptides by methods known in the art. Labeling of peptide mimetics usually involves covalent attachment of one or more labels, directly or through a spacer (e.g., an amide group), to non-interfering positions on the peptide mimetic that are predicted by quantitative structure-activity data and molecular modeling. Such non-interfering positions generally are positions that do not form direct contacts with the macromolecules to which the peptide mimetic binds to produce the therapeutic effect. Derivitization (e.g., labeling) of peptide mimetics should not substantially interfere with the desired biological or pharmacological activity of the peptide mimetic.

The use of peptide mimetics can be enhanced through the use of combinatorial chemistry to create drug libraries. The design of peptide mimetics can be aided by identifying amino acid mutations that increase or decrease binding of a peptide to, for instance, a tumor cell. Approaches that can be used include the yeast two hybrid method (see Chien et al. (1991) Proc. Natl. Acad. Sci. USA 88, 9578-9582) and using the phage display method. The two hybrid method detects protein-protein interactions in yeast (Fields et al. (1989) Nature 340, 245-246). The phage display method detects the interaction between an immobilized protein and a protein that is expressed on the surface of phages such as lambda and M13 (Amberg et al. (1993) Strategies 6, 2-4; Hogrefe et al. (1993) Gene 128, 119-126). These methods allow positive and negative selection for peptide-protein interactions and the identification of the sequences that determine these interactions.

### Recombinant DNA Molecules Containing a Nucleic Acid Molecule

The present invention further provides recombinant DNA molecules (rDNA) that contain a coding sequence. As used herein, a rDNA molecule is a DNA molecule that has been subjected to molecular manipulation in situ. Methods for generating rDNA molecules are well known in the art, for example, see Sambrook et al. (2001) Molecular Cloning—A Laboratory Manual, Cold Spring Harbor Laboratory Press. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably, also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 (BioRad), pPL and pKK223 (Pharmacia).

Expression vectors compatible with eukaryotic cells, preferably those compatible with vertebrate cells, can also be used to form rDNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC), the vector pCDM8 described herein, and the like eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al. (1982) J. Mol. Anal. Genet. 1, 327-341). Alternatively, the selectable marker can be present on a separate plasmid, and the two vectors are introduced by co-transfection of the host cell, and selected by culturing in the appropriate drug for the selectable marker. The present invention further provides host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells (NIH-3T3) available from the ATCC as CRL 1658, baby hamster kidney cells (BHK), and the like eukaryotic tissue culture cell lines.

Any prokaryotic host can be used to express a rDNA molecule encoding a protein of the invention. The preferred prokaryotic host is E. coli.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al. (1972) Proc. Natl. Acad. Sci. USA 69, 2110; and Sambrook et al. (1989) Molecular —A Laboratory Manual, Cold Spring Harbor Laboratory Press. With regard to transformation of vertebrate cells with vectors containing rDNAs, electroporation, cationic lipid or salt treatment methods are typically employed, see, for example, Graham et al. (1973) Virol. 52, 456; Wigler et al. (1979) Proc. Natl. Acad. Sci. USA 76, 1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern (1975) J. Mol. Biol. 98, 503-504 or Berent et al. (1985) Biotech. 3, 208-209 or the proteins produced from the cell assayed via an immunological method.

Production of Recombinant Proteins Using a rDNA Molecule

The present invention further provides methods for producing a protein of the invention using nucleic acid molecules herein described. In general terms, the production of a recombinant form of a protein typically involves the following steps:

A nucleic acid molecule is first obtained that encodes a protein of the invention, such as a nucleic acid molecule comprising, consisting essentially of or consisting of the nucleic acid sequence encoding SEQ ID NO: 8 or derivatives thereof.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

### Methods to Identify Binding Partners

Another embodiment of the present invention provides methods for use in isolating and identifying binding partners of proteins of the invention. In general, a protein of the invention is mixed with a potential binding partner or an extract or fraction of a cell under conditions that allow the association of potential binding partners with the protein of the invention. After mixing, peptides, polypeptides, proteins or other molecules that have become associated with a protein of the invention are separated from the mixture. The binding partner that bound to the protein of the invention can then be removed and further analyzed. To identify and isolate a binding partner, the entire protein, for instance a protein comprising the entire amino acid sequence of SEQ ID NO: 8 can be used. Alternatively, a fragment of the protein can be used.

As used herein, a cellular extract refers to a preparation or fraction that is made from a lysed or disrupted cell. The preferred source of cellular extracts will be cells derived from human skin tissue or the human respiratory tract or cells derived from a biopsy sample of human lung tissue in patients with allergic hypersensitivity. Alternatively, cellular extracts may be prepared from normal tissue or available cell lines, particularly granulocytic cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the protein of the invention under conditions in which association of the protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture. To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract. The identified binding partners can be either a single protein or a complex made up of two or more proteins. Alternatively, binding partners may be identified using a Far-Western assay according to the procedures of Takayama et al. (1997) Methods Mol. Biol. 69, 171-184 or Sauder et al. (1996) J. Gen. Virol. 77, 991-996 or identified through the use of epitope tagged proteins or GST fusion proteins.

Alternatively, the nucleic acid molecules of the invention can be used in a yeast two-hybrid system. The yeast two-hybrid system has been used to identify other protein partner pairs and can readily be adapted to employ the nucleic acid molecules herein described.

Methods to Identify Agents that Modulate Activity

The present invention provides methods for identifying agents that modulate at least one activity of a protein of SEQ ID NO: 8. Such methods or assays may utilize any means of monitoring or detecting the desired activity. These assays may include detection of the acetylation of S6K2 or the monitoring of the ability of S6K2 to phosphorylate a second protein.

In one format, the specific activity of a protein of the invention, normalized to a standard unit, between a cell population that has been exposed to the agent to be tested compared to an un-exposed control cell population may be assayed. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) Biotechnology 24, 524-526 or modifications which effect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies may be recovered from the culture supernatant or from the ascites supernatant. Fragments of the monoclonal antibodies or the polyclonal antisera that contain the immunologically significant portion can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin.

Antibody regions that bind specifically to the desired regions of the protein can also be produced in the context of chimeras with multiple species origin, for instance, humanized antibodies. The antibody can therefore be a humanized antibody or human a antibody, as described in U.S. Pat. No. 5,585,089 or Riechmann et al. (1988) Nature 332, 323-327.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies or fragments thereof that bind to a protein of SEQ ID NO: 8. Antibody agents can be obtained by immunization of suitable mammalian subjects with peptides, containing as antigenic regions, those portions of the protein intended to be targeted by the antibodies.

Methods to Identify Agents that Modulate the Expression

Another embodiment of the present invention provides methods for identifying agents that modulate the expression of a nucleic acid encoding an 56 kinase protein such as a protein having the amino acid sequence of SEQ ID NO: 8. Such assays may utilize any available means of monitoring for changes in the expression level of the nucleic acids of the invention. As used herein, an agent is said to modulate the expression of a nucleic acid of the invention, for instance a nucleic acid encoding the protein having the sequence of SEQ ID NO: 8, if it is capable of up- or down-regulating expression of the nucleic acid in a cell.

In one assay format, cell lines that contain reporter gene fusions between the open reading frame of the S6K2 (SEQ ID NO: 7) and any assayable fusion partner may be prepared. Numerous assayable fusion partners are known and readily available including the firefly luciferase gene and the gene encoding chloramphenicol acetyltransferase (Alam et al., 1990 Anal. Biochem. 188, 245-254). Cell lines containing the reporter gene fusions are then exposed to the agent or a polypeptide comprising a p300 acetyltransferase binding domain (i.e., control) to be tested under appropriate conditions and time. Differential expression of the reporter gene between samples exposed to the agent and control samples identifies agents which modulate the expression of a nucleic acid encoding the S6 kinase protein.

Additional assay formats may be used to monitor the ability of the agent to modulate the expression of a nucleic acid encoding a protein of the invention such as the S6 kinase protein. For instance, mRNA expression may be monitored directly by hybridization to the nucleic acids of the invention. Cell lines are exposed to the agent and/or a polypeptide comprising a p300 acetyltransferase binding domain to be tested under appropriate conditions and time and total RNA or mRNA is isolated by standard procedures such those disclosed in Sambrook et al. (2001) Molecular Cloning, A Laboratory Manual, Cold Spring Harbor Laboratory Press).

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al. (2001) or Ausubel et al. (1995) Current Protocols in Molecular Biology, Greene Publishing Company.

Hybridization conditions are modified using known methods, such as those described by Sambrook et al. (2001) and Ausubel et al. (1995) as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a porous glass wafer. The glass wafer can then be exposed to total cellular RNA or polyA RNA from a sample under conditions in which the affixed sequences will specifically hybridize. Such glass wafers and hybridization methods are widely available, for example, those disclosed by Beattie (WO 95/11755). By examining for the ability of a given probe to specifically hybridize to an RNA sample from an untreated cell population and from a cell population exposed to the agent, agents which up or down regulate the expression of a nucleic acid encoding the S6 kinase protein.

### Pharmaceutical Compositions

This invention includes a pharmaceutical composition for the treatment of abnormal cell growth or for a disease characterized by cell proliferation in a mammal, including a human, comprising, consisting essentially of, or consisting of an amount of polypeptide comprising p300 acetyltransferase binding domain or derivatives thereof that is effective in inhibiting an S6 kinase protein mediated activity and a pharmaceutically acceptable carrier. In one embodiment, the S6 kinase protein mediated activity is abnormal cell growth. As used herein, the terms “abnormal cell growth” and “disease characterized by cell proliferation” unless otherwise indicated, refer to cell growth or proliferation that is independent of normal regulatory mechanisms (e.g., loss of contact inhibition). This includes the abnormal growth and/or proliferation of cells in both benign and malignant cells of neoplastic diseases. Inhibition of abnormal cell growth can occur by a variety of mechanisms including, but not limited to, cell death, apoplosis, arrest of mitosis, inhibition of cell division, transcription, translation, transduction, etc.

As used herein, an “effective amount” of p300 acetyltransferase binding domain fragment is an amount which exerts an inhibitory effect on S6 kinase protein activity, including phosphorylation of a substrate protein. An effective amount is also an amount which is effective to inhibit the proliferation of cells exhibiting abnormal growth, such as cancer cells. As used herein, compositions comprising, treatment with or administration of a polypeptide containing a “p300 acetyltransferase binding domain” includes to the same degree treatment with analogues, derivatives, fragments, variants, related peptides and mimetics disclosed herein.

Pharmaceutical compositions comprising the active agents (e.g., polypeptides, small molecules, etc.) of the present invention can be administered via parenteral, subcutaneous, intravenous, intramuscular, intraperitoneal, intrathecal, intracranial or transdermal or buccal routes. For example, an agent may be administered locally to a tumor via microinfusion. Alternatively, or concurrently, administration may be by the oral route. The dosage administered will be dependent upon the age, health, and weight of the recipient, kind of concurrent treatment, if any, frequency of treatment, and the nature of the effect desired.

While individual needs vary, determination of optimal ranges of effective amounts of each component is within the skill of the art. Dosages of p300-4 fragment and/or derivatives thereof of the present invention typically comprise about 1.0 ng/kg body weight to about 0.13 mg/kg body weight. In one embodiment, dosages of p300-4 fragment and/or derivatives thereof comprise about 1.0 ng/kg body weight to about 0.1 mg/kg body weight. In a preferred embodiment, dosages for systemic administration comprise about 0.01 μg/kg body weight to about 0.1 mg/kg body weight. In another embodiment, the dosage of p300-4 fragment and/or derivatives thereof comprises less than about 0.1 mg/kg body weight. More preferred dosages for systemic administration comprise about 0.1 μg/kg body weight to about 0.05 mg/kg body weight. In another preferred embodiment, the dosage of p300-4 fragment and/or derivatives thereof comprises less than about 0.05 mg/kg body weight. The most preferred dosages for systemic administration comprise between about 1.0 μg/kg body weight to about 0.01 mg/kg body weight. In other embodiments, the amount of p300-4 fragment and/or derivatives thereof administered is an amount effective to bring the concentration of p300-4 fragment and/or derivatives thereof in the serum to a concentration of about 20.0, 10.0, 5.0, 2.50, 1.25, 0.625, 0.3125, 0.156, 0.078, 0.039, 0.020, 0.010, 0.005, 0.003, 0.0015, 0.0008, 0.0003, or 0.0001 nM.

Dosages of S6K2/or derivatives thereof of the present invention typically comprise about 1.0 ng/kg body weight to about 0.13 mg/kg body weight. In one embodiment, dosages of S6K2 and/or derivatives thereof comprise about 1.0 ng/kg body weight to about 0.1 mg/kg body weight. In a preferred embodiment, dosages for systemic administration comprise about 0.01 μg/kg body weight to about 0.1 mg/kg body weight. In another embodiment, the dosage of S6K2 and/or derivatives thereof comprises less than about 0.1 mg/kg body weight. More preferred dosages for systemic administration comprise about 0.1 mg/kg body weight to about 0.05 mg/kg body weight. In another preferred embodiment, the dosage of S6K2 and/or derivatives thereof comprises less than about 0.05 mg/kg body weight. The most preferred dosages for systemic administration comprise between about 1.0 mg/kg body weight to about 0.01 mg/kg body weight. In other embodiments, the amount of S6K2 and/or derivatives thereof administered is an amount effective to bring the concentration of S6K2 and/or derivatives thereof in the serum to a concentration of about 20.0, 10.0, 5.0, 2.50, 1.25, 0.625, 0.3125, 0.156, 0.078, 0.039, 0.020, 0.010, 0.005, 0.003, 0.0015, 0.0008, 0.0003 or 0.0001 nM. The preferred dosages for direct administration to a site via microinfusion comprise 1 ng/kg body weight to 1 mg/kg body weight.

In addition to S6K2 polypeptide or derivatives thereof or in the alternative to p300 acetyltransferase binding domain polypeptides or derivatives thereof, the pharmaceutical compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

As mentioned above for some methods of the invention, topical administration may be used. Any common topical formulation such as a solution, suspension, gel, ointment or salve and the like may be employed. Preparation of such topical formulations are described in the art of pharmaceutical formulations as exemplified, for example, by Gennaro et al. (2000) Remington's Pharmaceutical Sciences, Mack Publishing. For topical application, the compositions could also be administered as a powder or spray, particularly in aerosol form. In some embodiments, the compositions of this invention may be administered by inhalation. For inhalation therapy, the active ingredients may be in a solution useful for administration by metered dose inhalers or in a form suitable for a dry powder inhaler. In another embodiment, the compositions are suitable for administration by bronchial lavage.

Suitable formulations for oral administration include hard or soft gelatin capsules, pills, tablets, including coated tablets, elixirs, suspensions, syrups or inhalations and controlled release forms thereof.

The invention also includes isotopically-labeled agents that bind to S6 kinase, including human S6 kinase, that have one or more atoms replaced by an atom having an atomic mass or mass number different from the atomic mass or mass number usually found in nature. In one embodiment, the isotopically labeled agents bind to the acetylated form of S6 kinase while in other embodiments, they bind to the deacetylated form of S6 kinase.

In one embodiment of the composition and methods of the invention, the abnormal cell growth is cancer. As used herein, the term “cancer” unless otherwise indicated, refers to diseases that are characterized by uncontrolled, abnormal cell growth and/or proliferation. Types of cancer where the compositions are useful include, but are not limited to, prostate cancer, breast cancer, multiple myeloma, lung cancer, non-small cell lung carcinoma, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancers, spinal axis tumors, glioma, meningioma, pituitary adenoma, or a combination of one or more of the foregoing cancers.

In another embodiment of the compositions and methods of the invention, the abnormal cell growth is a benign proliferative disease, including, but not limited to, benign prostatic hyperplasia, hypertrophy or restinosis.

As discussed above, the invention includes compositions and methods for the treatment of abnormal cell growth in a mammal, including a human, which comprises, consists of, or consists essentially of an effective amount of a polypeptide containing a p300 acetyltransferase binding domain, in combination with a pharmaceutically acceptable carrier.

The invention further includes isotopically-labeled agents that bind to S6K2 at the AT-hook domain, including human S6K2, that have one or more atoms replaced by an atom having an atomic mass or mass number different from the atomic mass or mass number usually found in nature. In one embodiment, the isotopically-labeled agents bind to the activated form of S6K2 while in other embodiments, they bind to the inactivated form of S6K2.

Examples of isotopes that can be incorporated into compounds of the invention include, but are not limited to, isotopes of hydrogen, carbon, phosphorous, iodine, rhenium, indium, yttrium, technetium and lutetium (i.e., including, but not limited to, ³H, ¹⁴C, ³¹P, ³²P, ³⁵S, ¹³¹I, ¹²⁵I, ¹²³I, ¹⁸⁷Re, ⁶⁴Cu, ¹¹¹In, ⁹⁰Y, ^{99m}Tc, ¹⁷⁷Lu), others isotopes of these elements, and other isotopes known in the art. Agents of the present invention, prodrugs thereof, and pharmaceutically acceptable salts of said agents or of said prodrugs which contain the aforementioned isotopes and/or other isotopes of other atoms are within the scope of this invention. Tritium and carbon-14 isotopes are particularly preferred for their ease of preparation and detectability. Further, substitution with heavier isotopes such as deuterium can afford certain therapeutic advantages resulting from greater metabolic stability, for example increased in vivo half-life or reduced dosage requirements and, hence, may be preferred in some circumstances.

### Fusion Proteins

The invention includes fusion polypeptides containing a p300 acetyltransferase binding domain and salts thereof, comprising at least one second polypeptide. In one embodiment, the fusion polypeptides are capable of binding to S6 kinase, including human S6 kinase. In a further embodiment, the fusion polypeptides are capable of modulating the activity of the S6 kinase by effecting acetylation at the binding domain. In one embodiment, the fusion polypeptides facilitate acetylation while in another embodiment, they inhibit acetylation by another acetyltransferase (e.g., p300).

In some embodiments, the second polypeptide includes a cancer cell-binding domain which specifically binds to an epitope expressed only on cells exhibiting abnormal growth (i.e. cancer cells). The term “cancer cell-binding domain” refers to an amino acid sequence capable of binding or otherwise specifically associating with a cell displaying abnormal growth (e.g., benign and malignant cancer cells). In some embodiments the cancer cell binding-domain is an antibody while in other embodiments it is a ligand which specifically binds to a receptor expressed only on cancer cells. Examples of antibodies include, but are not limited to, antibodies which specifically bind to B-cells or T-cells. Examples of receptor ligands include, but are not limited to, cytokines and growth factors including epidermal growth factor.

The second polypeptide can also include a stabilization domain which increases the in vitro and in vivo half-life of the fusion polypeptide. As used herein, the term “stabilization domain” refers to an amino acid sequence capable of extending the in vitro and in vivo half-life of the p300 acetyltransferase binding domain polypeptide when compared to polypeptide alone. The stabilization domain can comprise human proteins (e.g., full length or truncated, soluble proteins from extracellular fragments, etc) such as human serum albumin, transferrin or other proteins which stabilize the in vivo or in vitro half-life of the polypeptide or derivative thereof. These additional functional domains may themselves serve as linker peptides, for example, for joining a cancer cell-binding domain to the p300-4 fragment or derivative. Alternatively, they may be located elsewhere in the fusion molecule (e.g., at the amino or carboxy terminus thereof). In alternative embodiments, the stabilization domain is a chemical moiety (e.g., PEG (polyethylene glycol) or a dextran).

The term “fused” or “fusion polypeptide” as used herein refers to polypeptides in which: (i) a given functional domain (i.e. a cancer cell-binding domain) is bound at its carboxy terminus by a covalent bond either to the amino terminus of another functional domain (i.e., an human serum albumin component) or to a linker peptide which itself is bound by a covalent bond to the amino terminus of p300 acetyltransferase binding domain; or (ii) a given functional domain (i.e. a cancer cell-binding domain) is bound at its amino terminus by a covalent bond either to the carboxy terminus of another functional domain (i.e., an human serum albumin component) or to a linker peptide which itself is bound by a covalent bond to the carboxy terminus of the p300 acetyltransferase binding domain.

Similarly, “fused” when used in connection with the nucleic acid intermediates of the invention means that the 3′-[or 5′-] terminus of a nucleotide sequence encoding a first functional domain is bound to the respective 3′-[or 5′-] terminus of a nucleotide sequence encoding a second functional domain, either by a covalent bond or indirectly via a nucleotide linker which itself is covalently bound preferably at its termini to the first functional domain-encoding polynucleotide and optionally, a second functional domain-encoding nucleic acid.

Examples of fusion polypeptides of the invention may be represented by, but are not limited by, the following formulas:

R1-L-R2 (i)

R2-L-R1 (ii)

R1-L-R2-L-R1 (iii)

R1-L-R1-L-R2 (iv)

R2-L-R1-L-R1 (v)

wherein R1 is the amino acid sequence of a cancer cell-binding domain, R2 is the amino acid sequence of a stabilizing domain (e.g., human serum albumin), each L is p300 acetyltransferase binding domain which is bound by a covalent bond to a terminus of R1 and/or R2, whereby the above molecule fragments are read directionally (i.e., with the left side corresponding to the amino terminus and the right side to the carboxy terminus of the molecule).

The present invention also includes compositions where a cytotoxic agent is linked to a p300 acetyltransferase binding domain polypeptide or derivative thereof. Examples of cytotoxic agents include, but are not limited to, gelonin, ricin, saponin, pseudonomas exotoxin, pokeweed antiviral protein, diphtheria toxin, complement proteins, or any other agent known in the art which is capable of killing a cell upon contact with that cell.

### Methods of Treatment

This invention includes methods for the treatment of abnormal cell growth in a mammal, including a human, comprising administering to said mammal an amount of an agent capable of modulating the acetylation of a kinase protein. In one embodiment, the acetylation is inhibited while in another embodiment the agent is capable of acetylating, or facilitating the acetylation of, the kinase protein. In another embodiment, the kinase protein is S6 kinase, including human S6 kinase. This invention also includes methods for the treatment of abnormal cell growth in a mammal, including a human, comprising administering to said mammal an amount of an agent capable of modulating the activity of a kinase protein. In one embodiment, the DNA binding is inhibited while in another embodiment the agent is capable of facilitating the DNA binding of a kinase protein. In another embodiment, the kinase protein is S6K2, including human S6K2.

In one aspect of the invention, the above described agents are deacetylase inhibitors which can be administered to inhibit the activity of a kinase which is modulated by an acetyltransferase. In one embodiment, the deacetylase inhibitor is an S6 kinase deacetylase inhibitor. In another embodiment, the deacetylase inhibitor is an PKCa kinase deacetylase inhibitor. In yet another embodiment, the deacetylase inhibitors will have the same activity as the p300-4 fragment described herein (i.e., inhibition of S6 kinase activity). Types of deacetylase inhibitors which can be used in the methods of the invention include, but are not limited to, low molecular weight carboxylates (e.g., sodium butyrate, valproic acid, sodium phenylbutyrate), hydroxamic acids (e.g., CBHA, TSA, SAHA, LAQ824), benzamides (e.g., MS-275, CI-994), epoxyketones (e.g., AOE, trapoxin B), cyclic peptides (e.g., depsipeptide, apicidin), and hybrid molecules such as CHAP31 and CHAP50. Exemplary examples of deacetylase inhibitors include, but are not limited to, hydroxamate-type HDAC inhibitors such as LAQ824 (Atadja et al. (2004) Cancer Res. 64, 689-695), suberoylanilide hydroxamic acid (Kelly et al. (2003) Clin. Cancer Res. 9, 3578-3588); CI-994 (Loprevite et al. (2005) Oncol Res. 15, 39-48); JNJ1641199 (Arts et al. (2003) AACR-NCI-EORTC Int. Conf. Mol. Targets. Cancer Ther.) and those set forth in Guo et al. (2005) Ann. Rev. Pharmacol. Toxicol. 45, 495-528).

The invention also includes methods of treatment of the above referenced kinase-related disorders with a deacetylase inhibitor in combination with kinase inhibitor. Types of kinase inhibitors which can be used in the methods of the invention include, but are not limited to, rampamycin derivatives such as RAD001 (Lane (2003) Mol. Targets. Cancer Therapeut. 259-260); serine-threonine kinase inhibitors such as mTor inhibitors, including but not limited to, AP23573 (Mita et al. (2004) Proc. American Soc. Clin. Oncology, 23, 3076) and CCI-779 (Yu et al. (2001) Endocrine-Related Cancer, 8, 249-258). In one embodiment, the deacetylase inhibitor is an S6 kinase inhibitor.

In some embodiments of the invention, the agent will have activity similar any of the following: a polypeptide comprising p300 acetyltransferase binding domain or derivative thereof, an fusion protein comprising p300 acetyltransferase binding domain or a derivative thereof, or a pharmaceutical composition comprising an amount of p300 acetyltransferase binding domain or derivatives thereof, that is effective in inhibiting S6 kinase mediated activity associated with abnormal cell growth, without the addition of other therapeutic agents.

In some embodiments of this method, the abnormal cell growth is cancer, including, but not limited to, prostate cancer, breast cancer, multiple myeloma, lung cancer, non-small cell lung carcinoma, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma, pituitary adenoma, or a combination of one or more of the foregoing cancers. In another embodiment of said method, said abnormal cell growth is a benign proliferative disease, including, but not limited to, psoriasis, benign prostatic hyperplasia, hypertrophy or restinosis.

This invention also includes methods for the treatment of abnormal cell growth in a mammal which comprises administering to said mammal, including a human, a pharmaceutical composition comprising an amount of an agent capable of modulating the acetylation of a kinase protein. The methods may alternatively comprise administration of an amount of agent capable of modulating the DNA binding of a kinase protein. In one embodiment, the acetylation is inhibited while in another embodiment the agent itself is capable of acetylating the kinase protein. In an alternate embodiment, the DNA binding is inhibited while in another embodiment the agent itself is capable of facilitating DNA binding by the kinase protein. In another embodiment, the kinase protein is S6 kinase, including human S6 kinase. In yet another embodiment, the kinase protein is S6K2, including human S6K2. In some embodiments, the agent is a polypeptide comprising a p300 acetyltransferase binding domain or derivatives thereof that is effective in inhibiting abnormal cell growth associate with S6 kinase expression and activity.

In some other embodiments, the agent is effective in inhibiting abnormal cell growth associate with S6K2 expression and activity. This includes the abnormal growth and/or proliferation of cancer cells including benign and malignant cells of neoplastic diseases. Inhibition of abnormal cell growth can occur by a variety of mechanisms including, but not limited to, cell death, apoptosis, inhibition of cell division, transcription, translation, transduction, etc.

In practicing the methods of this invention, these agents may be used alone or in combination with other inactive ingredients. As discussed above, the present invention includes compositions and methods where a drug or cytotoxic agent is linked to an agent capable of modulating acetylation of a kinase, including S6 kinase. In addition, as discussed above, the present invention includes compositions and methods where a drug or cytotoxic agent is linked to an agent capable of modulating DNA binding of a kinase, including S6K2. The methods of the invention therefore include administration of a polypeptide comprising these agents linked to a cytotoxic agent for the treatment of a disease associated with abnormal cell growth, including cancer. Examples of cytotoxic agents include, but are not limited to, gelonin, ricin, saponin, pseudonomas exotoxin, pokeweed antiviral protein, diphtheria toxin, complement proteins, or any other agent known in the art which is capable of killing a cell upon contact with that cell.

The compositions and methods of the invention for therapeutic purposes can be utilized in vivo, ordinarily in mammals, such as humans, sheep, horses, cattle, pigs, dogs, cats, rats and mice or ex vivo. The invention is particularly useful in the treatment of human subjects.

### Diagnostic Methods

This invention includes methods for the diagnosis of a disorder associated with abnormal kinase activity, including cancer, in a mammal, including a human, comprising administering to said mammal an effective amount of antibody which is capable of measuring acetylation of one or more kinases in a cell, such as a cancer cell. The diagnostic methods of the invention include in vitro methods where a biological sample is first isolated from a mammal, including humans, and then contacted with an amount of the antibody, that is effective in specifically and selectively binding to cells expressing an acetylated form of a kinase, such as cancer cells.

In one embodiment of this diagnostic method, the kinase related disorder is cancer. Types of cancer included in the methods of the invention include, but are not limited to, prostate cancer, breast cancer, multiple myeloma, lung cancer, non-small cell lung carcinoma, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma, pituitary adenoma, or a combination of one or more of the foregoing cancers. In another embodiment of said method, said abnormal cell growth is a benign proliferative disease, including, but not limited to, psoriasis, benign prostatic hyperplasia, hypertrophy or restinosis.

In practicing the diagnostic methods of this invention, the antibody may be used alone or in combination with other inactive ingredients. As discussed above, the present invention includes compositions and methods where a label is linked to the antibody. The methods of the invention therefore include administration of labeled antibody the diagnosis of a disease associated with abnormal kinase activity, including cancer.

The compositions and methods of the invention for diagnostic purposes can be utilized both in vivo, ordinarily in mammals, such as humans, sheep, horses, cattle, pigs, dogs, cats, rats and mice or in vitro. The invention is particularly useful in the diagnosis of human subjects.

Methods to Identify Agents that Modulate Activity

The present invention provides methods for identifying agents that modulate at least one activity of a kinase (e.g., phosphorylation) by measuring acetylation of the kinase protein by an acetyltransferase protein. Such methods or assays may utilize any means of monitoring or detecting the desired activity (i.e., acetylation) and include changes in phosphorylation activity following acetylation of the kinase protein.

In one format, the specific activity of particular kinase, normalized to a standard unit, between a cell population that has been exposed to the agent to be tested compared to an unexposed control cell population may be assayed. Cell lines or populations are exposed to the agent to be tested under appropriate conditions and time. In this format, probes such as specific antibodies are used to monitor the phosphorylation of the kinase protein substrates in the different cell populations. In another embodiment, acetylation of the kinase protein in each of the samples can also be measured to determine the effect on kinase activity. Cellular lysates may be prepared from the exposed cell line or population and a control, unexposed cell line or population. The cellular lysates are then analyzed with the probe.

Agents that are assayed in the above method can be randomly selected or rationally selected or designed. As used herein, an agent is said to be randomly selected when the agent is chosen randomly without considering the specific sequences involved in the association of the protein of the invention alone or with its associated substrates, binding partners, etc. An example of randomly selected agents is the use a chemical library or a peptide combinatorial library, or a growth broth of an organism.

The agents of the present invention can be, as examples, peptides, peptide mimetics, antibodies, antibody fragments, small molecules, vitamin derivatives, as well as carbohydrates. A skilled artisan can readily recognize that there is no limit as to the structural nature of the agents of the present invention. In one embodiment, the small molecules are deacetylase inhibitors as described herein.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies or fragments thereof that bind to the either the kinase protein or the acetyltransferase. Antibody agents can be obtained by immunization of suitable mammalian subjects with peptides, containing as antigenic regions, those portions of the protein intended to be targeted by the antibodies as described herein.

Methods to Identify Agents that Modulate the Expression

Another embodiment of the present invention provides methods for identifying agents that modulate the expression of a nucleic acid encoding an S6 kinase protein such as a protein having the amino acid sequence of SEQ ID NO: 2 or 8. Such assays may utilize any available means of monitoring for changes in the expression level of the nucleic acids of the invention. As used herein, an agent is said to modulate the expression of a nucleic acid of the invention, for instance a nucleic acid encoding the protein having the sequence of SEQ ID NO: 1, if it is capable of up- or down-regulating expression of the nucleic acid in a cell.

In one assay format, cell lines that contain reporter gene fusions between the open reading frame of the S6 kinase protein alpha (SEQ ID NO: 1) or S6 kinase protein beta (SEQ ID NO: 7) and any assayable fusion partner may be prepared. Numerous assayable fusion partners are known and readily available including the firefly luciferase gene and the gene encoding chloramphenicol acetyltransferase (Alam et al., 1990 Anal. Biochem. 188, 245-254). Cell lines containing the reporter gene fusions are then exposed to the agent or a polypeptide comprising a p300 acetyltransferase binding domain (i.e., control) to be tested under appropriate conditions and time. Differential expression of the reporter gene between samples exposed to the agent and control samples identifies agents which modulate the expression of a nucleic acid encoding the S6 kinase protein.

Additional assay formats may be used to monitor the ability of the agent to modulate the expression of a nucleic acid encoding a protein of the invention such as the S6 kinase protein. For instance, mRNA expression may be monitored directly by hybridization to the nucleic acids of the invention. Cell lines are exposed to the agent and/or a polypeptide comprising a p300 acetyltransferase binding domain to be tested under appropriate conditions and time and total RNA or mRNA is isolated by standard procedures such those disclosed in Sambrook et al. (2001) Molecular Cloning, A Laboratory Manual, Cold Spring Harbor Laboratory Press).

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al. (2001) or Ausubel et al (1995) Current Protocols in Molecular Biology, Greene Publishing Company).

Hybridization conditions are modified using known methods, such as those described by Sambrook et al. (2001) and Ausubel et al. (1995) as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a porous glass wafer. The glass wafer can then be exposed to total cellular RNA or polyA RNA from a sample under conditions in which the affixed sequences will specifically hybridize. Such glass wafers and hybridization methods are widely available, for example, those disclosed by Beattie (WO 95/11755). By examining for the ability of a given probe to specifically hybridize to an RNA sample from an untreated cell population and from a cell population exposed to the agent, agents which up or down regulate the expression of a nucleic acid encoding the S6 kinase protein.

### Methods to Identify Binding Partners

Another embodiment of the present invention provides methods for identifying agents that modulate at least one activity of S6 kinase protein. Such methods or assays may utilize any means of monitoring or detecting the desired activity.

In one format, the relative amounts of a S6 kinase protein between a cell population that has been exposed to the agent to be tested and/or a polypeptide comprising a p300 acetyltransferase binding domain compared to an unexposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

For example, fragments comprising lysine 516 of S6 kinase protein can be expressed in bacteria and used to search for agents which bind to these fragments. Fusion proteins, such as His-tag or GST fusion to the N- or C-terminal regions of S6 kinase protein comprising lysine 516 can be prepared for use as a S6 kinase protein fragment substrate. These fusion proteins can be coupled to Talon or Glutathione-Sepharose beads and then probed with cell lysates. Prior to lysis, the cells may be treated with rapamycin or other drugs which may modulate S6 kinase protein or proteins that interact with S6 kinase protein. Lysate proteins binding to the fusion proteins can be resolved by SDS-PAGE, isolated and identified by protein sequencing or mass spectroscopy, as is known in the art.

Kinase assays to measure the ability of the agent to modulate the kinase activity of a protein of the invention are widely available such as the assays disclosed by Mishima et al. (1996) J. Biochem. 119, 906-913) and Michnoff et al. (1986) J. Biol. Chem. 261, 8320-8326. Alternative assay formats include actin-myosin motility assays such as those disclosed by Kohama et al. (1996) TIPS 17, 284-287 or Warrick et al. (1987) Ann. Rev. Cell. Biol. 3, 379-421.

Agents that are assayed in the above method can be randomly selected or rationally selected or designed. As used herein, an agent is said to be randomly selected when the agent is chosen randomly without considering the specific sequences involved in the association of the protein of the invention alone or with its associated substrates, binding partners, etc. An example of randomly selected agents are agents present in a chemical library or a peptide combinatorial library, or agents present in the medium in which an organism has been grown.

As used herein, an agent is said to be rationally selected or designed when the agent is chosen on a non-random basis which takes into account the sequence of the target site and/or its conformation in connection with the agent's action. There are proposed binding sites for ATP/GTP and calmodulin as well as cAMP/cGMP kinase sites, TyrP sites and Ser/Thr kinase (catalytic) sites in the protein having SEQ ID NO: 2 or SEQ ID NO: 8. Agents can be rationally selected or rationally designed by utilizing the peptide sequences that make up these sites. For example, a rationally selected peptide agent can be a peptide whose amino acid sequence is identical to the ATP or calmodulin binding sites or domains.

The agents of the present invention can be, as examples, peptides, small molecules, vitamin derivatives, as well as carbohydrates. A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

### Antibodies

Another class of agents of the present invention are antibodies. These antibodies may be immunoreactive with critical positions of S6 kinase protein where acetylation occurs (e.g., Lys516). In an alternate embodiment, these antibodies are immunoreactive with critical positions of the S6K2 protein where DNA binding occurs (e.g. amino acids corresponding to position 487 to 495 of SEQ ID NO: 8). Antibody agents are obtained by immunization of suitable mammalian subjects with peptides, containing as antigenic regions, those portions of the protein intended to be targeted by the antibodies.

Antibodies which bind to an acetylation site on a kinase protein are encompassed in the invention. Such antibodies include, but are not limited to, antibodies which bind to the acetylation site on the S6 kinase protein (i.e., Lysine at 516). Antibodies which also bind to the acetyltransferase domain on an acetyltransferase protein capable of acetylating a kinase are also encompassed in the scope of the invention. Such antibodies include, but are not limited to, antibodies which bind to the acetylation site on p300 acetyltransferase (i.e., residues 1,459 to 1,892), and capable of inhibiting the activity of this protein are also encompassed in the invention.

Antibodies are prepared by immunizing suitable mammalian hosts in appropriate immunization protocols using the peptides, polypeptides, or proteins of the invention, such as S6 kinase protein, variants and isolated binding partners, if they are of sufficient length, or, if desired, or if required to enhance immunogenicity, conjugated to suitable carriers. Methods for preparing immunogenic conjugates with carriers such as bovine serum albumin (BSA), keyhole limpet hemocyanin (KLH), or other carrier proteins are well known in the art. In some circumstances, direct conjugation using, for example, carbodiimide reagents may be effective; in other instances linking reagents well known in the art, may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

Anti-peptide antibodies can be generated using synthetic peptides corresponding to, for example, the carboxy terminal 15 amino acids S6 kinase protein or amino acids 487 to 495 of SEQ ID NO: 8. Antibodies which bind to the acetylation site on a kinase can also be generated and include antibodies which bind to the acetylation site on the S6 kinase protein (i.e., Lysine at 516). Synthetic peptides can be as small as 1 to 3 amino acids in length, but are preferably at least 4 or more amino acid residues long. The peptides are coupled to KLH using standard methods and can be immunized into animals such as rabbits. Polyclonal antibodies (e.g. anti-S6 kinase protein peptide, anti-S6K2 protein peptide) can then be purified, for example using Actigel beads containing the covalently bound peptide.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Milstein or modifications which effect immortalization of lymphocytes or spleen cells, as is generally known. The immortalized cell lines secreting the desired antibodies are screened by immunoassay in which the antigen is the peptide hapten. polypeptide or protein. When the appropriate immortalized cell culture secreting the desired antibody is identified, the cells can be cultured either in vitro or by production in ascites fluid. Of particular interest, are monoclonal antibodies which recognize the proline-rich domain of S6 kinase protein.

The desired monoclonal antibodies are then recovered from the culture supernatant or from the ascites supernatant. Fragments of the monoclonals or the polyclonal antisera which contain the immunologically significant portion can be used as antagonists, as well as the intact antibodies. Use of immunologically reactive fragments, such as Fav, _{SC}FV, Fab, Fab′ or F(ab′)₂ fragments is often preferable, especially in a therapeutic context, as these fragments are generally less immunogenic than the whole immunoglobulin. Antibodies may preferably also be human, humanized or chimeric variants of the foregoing. Such antibodies can be less immunogenic when administered to a subject. Methods of producing humanized or chimeric antibodies are well known in the art. The antibodies contemplated also include different isotypes and isotype subclasses (e.g., IgG₁, IgG₂, IgM). These antibodies can be prepared by raising them in vertebrates, in hybridoma cell lines or other cell lines, or by recombinant means. For references on how to prepare these antibodies, see Harlow & Lane (1988), Antibodies: A Laboratory Manual, Cold Spring Harbor Press.

The antibodies or fragments may also be produced, using current technology, by recombinant means. Regions that bind specifically to the desired regions of receptor can also be produced in the context of chimeras with multiple species origin.

Without further description, it is believed that one of ordinary skill in the art can, using the preceding description and the following illustrative examples, make and utilize the agents of the present invention and practice the claimed methods. The following working examples describe embodiments of the present invention, and are not to be construed as limiting in any way the remainder of the disclosure.

### Example 1

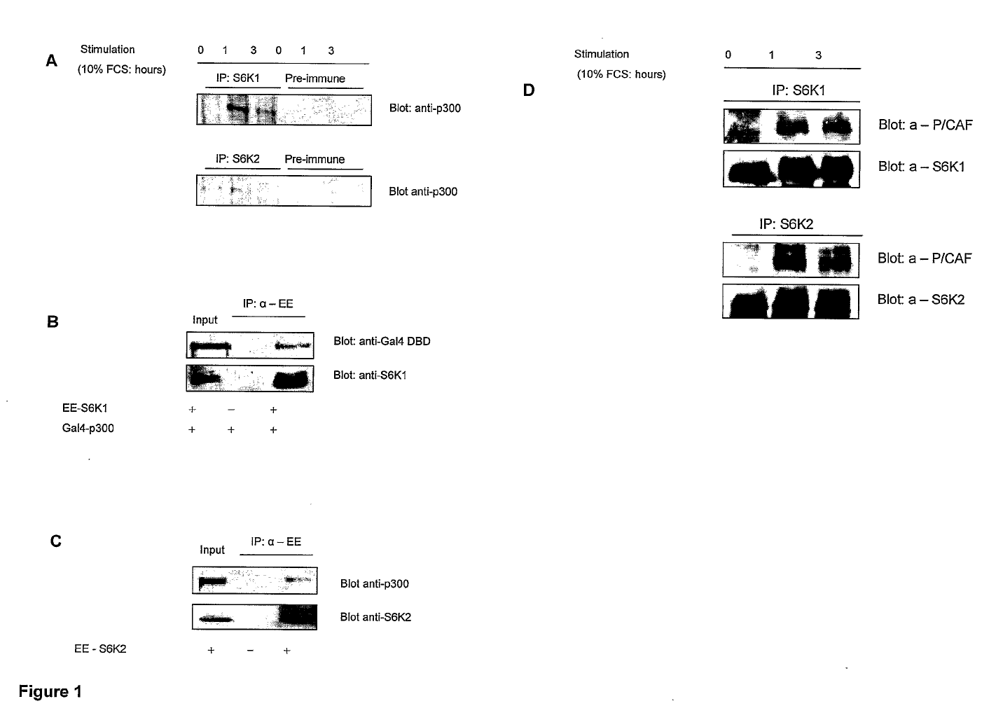

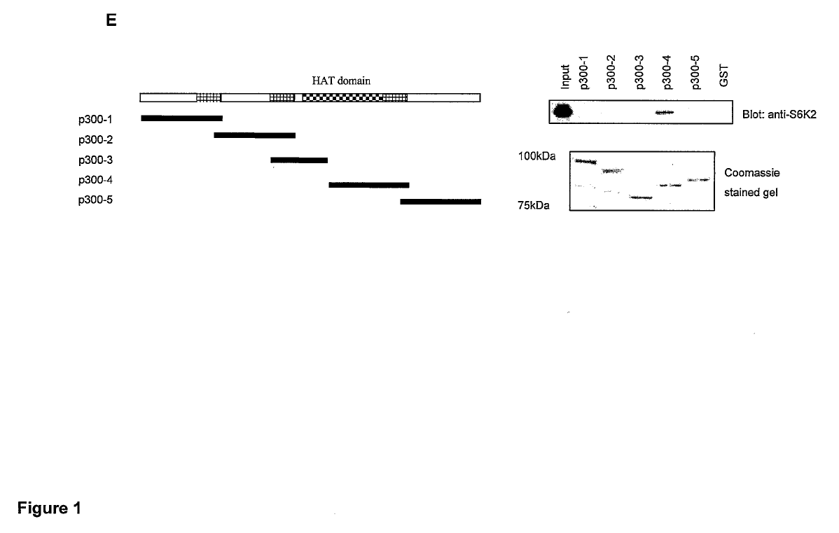

This example demonstrates that serum stimulation induces complex formation between p300 acetyltransferase and S6K1. Since S6 kinases appear to play a role in the regulation of transcription, it would be of interest to identify components of the transcriptional machinery with which they may interact. To this end, S6K1 and 2 were immunoprecipitated from MCF-7 cells before and after serum stimulation and blotting was carried out with a panel of antibodies to search for binding partners (data not shown). Interestingly, both isoforms of S6K show serum-inducible binding to the acetyltransferases, p300 and P/CAF (FIGS. 1A and D). Importantly, no p300 was detected in control immunoprecipitations using pre-immune sera from the rabbits used to produce the S6 kinase polyclonal antibodies. This demonstrates that the p300 interacts with the S6 kinases themselves rather than the anti-S6 kinase antibodies or protein A sepharose. The specificity of these interactions was further confirmed by overexpression of tagged S6 kinase and p300 constructs in HEK 293 and NIH-3T3 cells (FIGS. 1B and 1C). In FIG. 1B, a p300/Gal4 DNA binding domain (DBD) fusion was transfected into HEK 293 cells together with EE-tagged S6K1 or empty pcDNA 3.1 vector. EE-S6K1 was immunoprecipitated using anti-EE antibody and immunoprecipitates were resolved by SDS-PAGE. Following transfer, the top part of the nitrocellulose membrane was blotted with an antibody against the Gal4 DBD to detect coprecipitated p300, while the lower part was blotted with S6K1 antibodies. Note that lysates from cells transfected with p300 and the empty pcDNA 3.1 plasmid were also subjected to immunoprecipitation using the EE-antibody. The lack of a p300 band in this lane demonstrates that the p300 is binding specifically to S6K1 and not to the anti-EE antibody, or to the protein A sepharose. FIG. 1C shows binding of EE-tagged S6K2 to endogenous p300 in NIH 3T3 cells. Here, cells were transfected whether with EE-S6K2 or with empty pcDNA 3.1. Anti-EE immunoprecipitates were resolved by SDS-PAGE and this time, the blot was probed with p300 antibody. Again, p300 was only detected in the presence of S6K2 and not in the negative control anti-EE immunoprecipitate. In FIGS. 1B and 1C, cell lysate equal to approximately 10 percent of the input material used for immunoprecipitations was run alongside the samples (labeled ‘Input’). This gives an idea of the proportion of total p300 that is complexed with S6 kinases under these conditions and also serves as a marker to ensure the band detected in the IP lanes is of the correct molecular weight to be p300.

Having established that S6 kinases can interact with p300 in cells, it was interesting to see whether this interaction occurs directly and to determine to which region of p300 the S6 kinases bind. In order to investigate this, p300 was divided into five domains (FIG. 1D, upper panel). Each domain was cloned into the pGEX4T2 vector to allow expression as N-terminal GST fusion proteins in bacteria. These fusion proteins were expressed and purified and used for GST pulldown experiments with highly purified recombinant S6 kinases. FIG. 1D (lower panel) shows selective binding of S6K2 to p300 domain 4, which contains part of the acetyltransferase domain (FIG. 1D). Similar results were obtained for S6K1 (data not shown). These data show that S6 kinases can bind directly to p300 without the need for accessory factors such as other proteins or DNA, however, the interaction is weak, and is probably further stabilised in cells by other components.

### Example 2

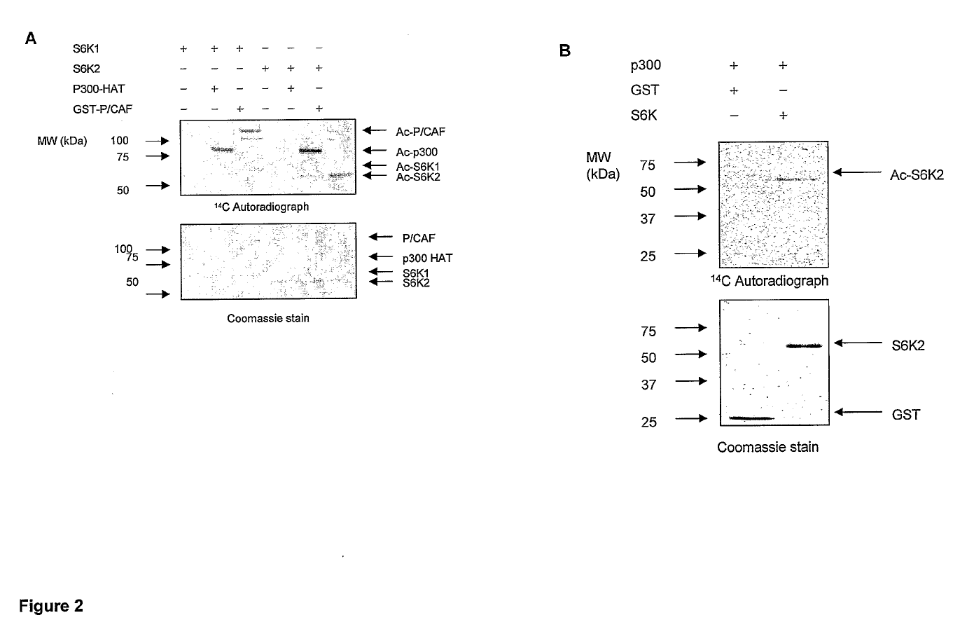

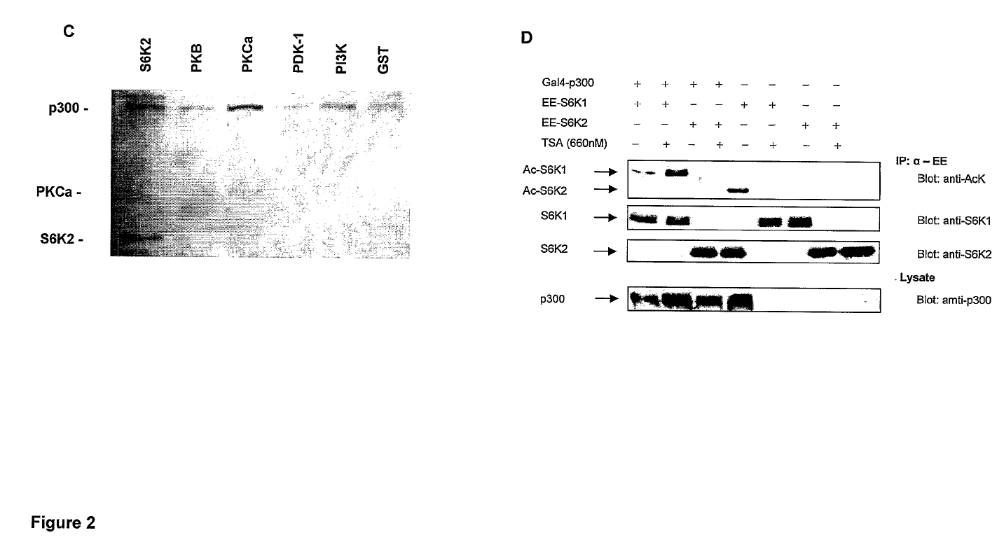

Since S6Ks can interact with a region within, or adjacent to the acetyltransferase domain of p300 we hypothesized that they may in fact be targeted by its enzymatic activity. This is indeed the case for two other proteins which bind to the p300-domain 4: Flap endonuclease-1 and DNA polymerase-β (Hasan et al. 2001b; Hasan et al. 2002) and for a number of proteins that bind to the third zinc finger region within domain 4, such as E1A, E2F, MyoD, SV40 large T and GATA-1 (Vo and Goodman, 2001). In order to test this possibility, recombinant S6K1 or 2 (1 μg) were incubated with GST-p300 HAT domain 4 or GST-P/CAF in HAT buffer containing ¹⁴C labelled acetyl coenzyme A (AcCoA). FIG. 2A shows that when S6K1 and S6K2 were incubated in the presence of GST-p300 HAT domain 4 or GST-P/CAF, ¹⁴C labeled acetyl groups were indeed incorporated into the proteins as visualized by autoradiographic detection of specific S6K bands following SDS-PAGE. Note that no label was incorporated into S6K1 or 2 in the absence of GST-p300 HAT domain 4 or GST-P/CAF (lanes 1 and 3). FIG. 2A shows that PCAF acetylates S6 kinases in vitro with a similar efficiency to that of the p300 HAT domain. In order to further test the specificity of S6K acetylation in vitro, acetylation reactions were conducted using recombinant S6K2 or GST (FIG. 2B). This experiment shows that while S6K2 is acetylated by p300, no acetylation of GST is seen under these conditions. Furthermore, in vitro acetylation of other recombinant proteins was tested, including protein kinases from AGC family. In order to test this possibility, full-length p300 was overexpressed in HEK 293 cells and immunoprecipitated. Recombinant kinases produced in insect cells (1 μg) were added to the immunoprecipitates in HAT buffer containing ¹⁴C labelled AcCoA. FIG. 2C shows that S6K2 is acetylated in the presence of p300 and AcCoA, but no acetylation is observed for PKB/Akt, PDK1, PI3-K (p85a/p110a complex) under conditions used. We detected weak, but reproducible acetylation of PKCa. These data further demonstrate the specificity of in vitro acetylation assay and indicate that other protein kinases could be acetylated in vitro.

In order to test whether S6 kinase proteins are subject to acetylation within cells, HEK 293 cells were cotransfected with EE-tagged S6 kinases and p300. Two days after transfection, cells were treated with Trichostatin A or control for 12 hours as indicated. Recombinant S6 kinases were immunoprecipitated using anti-EE and the immunocomplexes were subject to stringent washing to remove non-specifically bound proteins prior to SDS-PAGE. Immunoblotting was conducted with the anti-acetylated lysine antibodies from Upstate. FIG. 2D clearly shows that acetylated S6K1 and 2 can be detected in 293 cells in the presence of overexpressed p300. The level of S6 kinase acetylation was significantly increased in the presence of Trichostatin A (TSA). TSA is a specific inhibitor of class I and class II histone deacetylases (HDACs), which are responsible for the deacetylation of histones associated with gene silencing. Importantly, re-probing of blots following stripping of the acetyl lysine antibodies demonstrated similar levels of S6 kinases present in all lanes (FIG. 2D). This confirms the specificity of the acetyl lysine antibodies since they clearly bind to S6 kinases only when they are in an acetylated form. The finding that one or more of the class I/II HDACs targets S6 kinases in cells is in agreement with the published data on the other non-histone acetyltransferase substrates, which are also subject to deacetylation by these enzymes. Thus, it appears that S6 kinase acetylation (similarly to phosphorylation) is subject to dynamic regulation within the cell.

### Example 3

Having established that S6 kinases can be acetylated by p300 in vitro and in cells, attempts were made to find specific sites of acetylation in order to investigate the function(s) of S6 kinase acetylation. Recombinant S6 kinases were acetylated in vitro using either full length p300 or the recombinant p300 HAT domain. Reactions were carried out as before except that unlabelled AcCoA was used and the amount of S6 kinase was increased to 2 μg. S6 kinases were also incubated in the absence of p300 (termed un-acetylated samples). Reaction products were separated by SDS-PAGE, gels were stained with Coomassie blue and S6 kinase bands from both acetylated and un-acetylated samples were excised and subjected to in-gel digestion with trypsin or Arg-C prior to analysis by MALDI TOF mass spectroscopy.

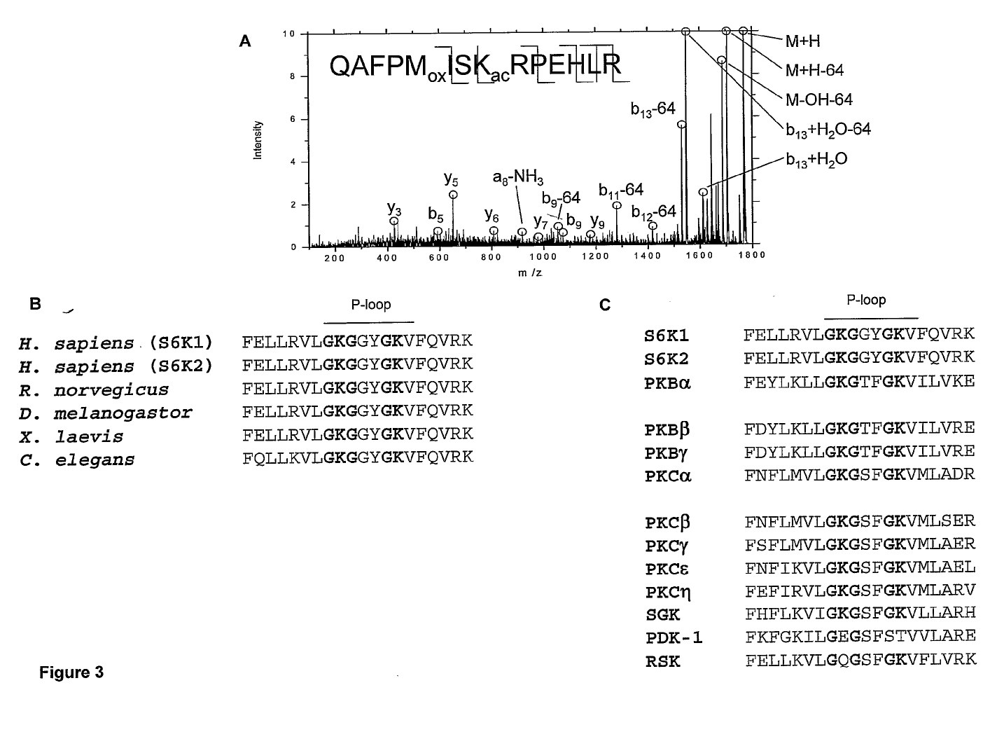

Initially, peptide mass fingerprints were acquired to check that the in-gel digestion was satisfactory, to establish the protein identification, and to look for putative acetylated peptide ion signals (data not shown). From the S6K1 sample treated with p300 and digested with trypsin, one ion signal was assigned to a tryptic acetylated S6K1 peptide. MALDI tandem mass spectrometry measurements using collision-induced dissociation unambiguously identified this ion as the 509Q-R522S6K1 peptide. The fragment ions of these measurements further revealed that this peptide was acetylated on the Lysine 516 (FIG. 3A).

In a complementary approach, samples were digested with Arg-C. This endoproteinase cleaves proteins exclusively at amide bonds directly C-terminal to arginine residues. Following digestion of S6K2 with this enzyme, two peptides predicted to contain acetylated lysines were detected from the acetylated sample. The first peptide, residues 297 to s310 contains a lysine at position 2 (residue 298 in S6K2). This peptide was sequenced and the acetylated lysine was confirmed (data not shown). A second peptide (residues 85 to 98) was predicted to be doubly acetylated. This peptide indeed contained two lysines (positions 88 and 93). It was not possible to sequence this peptide by MALDI tandem mass spectrometry so these lysines must be regarded as potential acetylation sites. It is important to note that both K88 and 93 in S6K2 are evolutionary conserved and are equivalent to K99 and K104 in S6K1.

TABLE 1
S6K Acetylation Sites

| | |
|---|---|
| Peptide (showing Ac-Lysine) | Enzyme |

| | |
|---|---|
| S6K1:K516 | Trypsin |
| ⁵⁰⁹ QAFPMISKRPEHLR⁵²² | |
| S6K2:K298 | |
| ²⁹⁷ GKLALPPYLTPDAR³¹⁰ | Arg-C |
| S6K2:K88,K93 | |
| ⁸⁵ VLGKGGYGKVFQVR⁹⁸ | Arg-C |

Alignment of the S6K1 and two amino acid sequences revealed that while K298 in S6K2 is present in S6K1, in this isoform, it is preceded by a cysteine rather than a glycine residue. Many published p300 acetylation sites conform to a consensus in which the target lysine is directly preceded by either a glycine or serine residue and this G/S-K motif has been suggested to be an optimal p300/CBP target sequence (Bannister et al. (2000) Curr. Biol. 10, 467-70). Note that all acetylation sites identified in the experiments presented here conform to this consensus (see Table 1). It is possible therefore, that the lysine in S6K1 equivalent to K298 in S6K2 is a poor substrate for p300 due to the preceding cysteine.

The sequence surrounding the double acetylation site, K88/93 is highly conserved between S6K2 and S6K1, suggesting that it could be acetylated in both kinases. Inspection of this region, located at the N-terminus of the kinase domain revealed that it is a feature highly conserved in all kinases (also found in other nucleotide binding proteins) called the P-loop. Alignment of this motif with S6Ks from other organisms revealed that both lysines are conserved throughout evolution, from C. elegans to humans (FIG. 3B). Comparison of the S6K P-loop with P-loops from other related AGC kinases showed that the lysines are highly conserved in all members of this family with the exception of PDK1 (FIG. 3B). The main feature of the P-loop is the invariant GXGXXG motif, which forms an ‘elbow’ over the bound ATP. The first glycine is in contact with the ribose moiety, while the second glycine lies close to the terminal pyrophosphate of the ATP (Hanks et al. (1987) Proc. Natl. Acad. Sci. USA 84, 388-392).

### Example 4

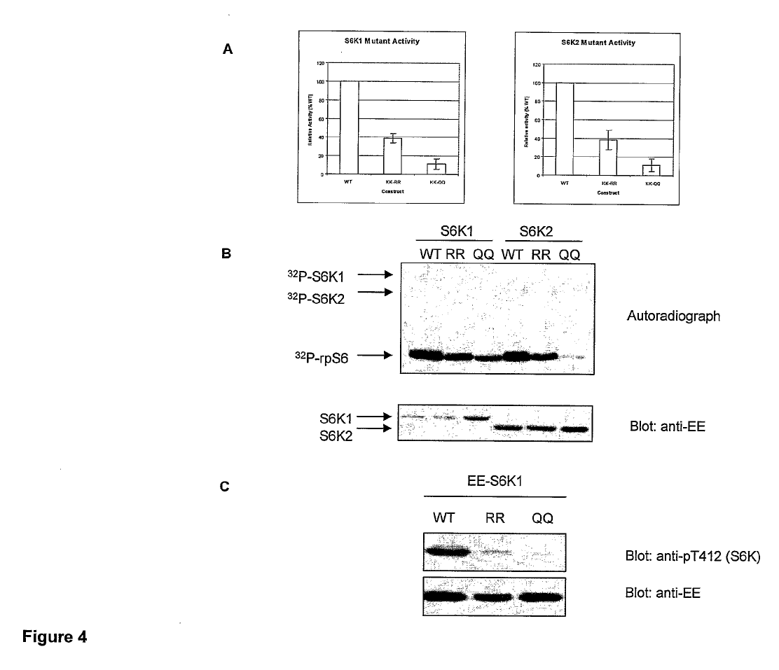

Mutation of the P-loop lysines severely abrogates S6K activity in vitro. Several recent reports have demonstrated that mutation of lysine to glutamine can mimic acetylation in a manner analogous to substitution of serines or threonines with acidic residues to mimic phosphorylation (Zhang et al. (1998) EMBO J. 17, 3155-3167; Gronroos et al. (2002) Mol. Cell. 10, 483-93). The glutamine residue is neutrally charged at physiological pH and contains a carbonyl group at the end of the side chain, two characteristics possessed by acetylated lysine. Arginine is most commonly substituted for lysine to produce a non-acetylated mutant since, like lysine, it possesses a basic side chain but cannot be acetylated. Since the P-loop is involved in ATP binding, we hypothesized that acetylation of the P-loop lysines may modulate the kinase activity of S6Ks in some way. In order to investigate this possibility, double mutants in which both P-loop lysines were replaced either with arginine or with glutamine were expressed in 293 cells and immunoprecipitated along with wild type S6Ks. Immune complex S6K assays were conducted and gels were exposed to a phosphorimager screen. These experiments were carried out on three separate occasions and in each case it was clear that mutation of these lysine residues has a significant inhibitory effect on the activity of S6Ks (FIGS. 4A and B). While the activity of the arginine mutant is significantly lower than that of the wild type, mutation to glutamine almost completely abrogates the S6K activity. These data suggest that while it is critical to have lysine at these positions (even the relatively subtle change to arginine reduces activity), acetylation of the lysines (mimicked by the glutamine mutant) may be a way in which S6K activity can be down-regulated by acetyltransferases. Note that phosphorylation of the critical rapamycin-sensitive S6K activation site, threonine 412 is much reduced in the P-loop mutants (FIG. 4C).

Taking these data into account, single-site mutants corresponding in S6K1 to K99 and K104. The wtS6K and both mutants were made, K99R and K104R, were cotransfected with p300 into Hek293 cells and immunoprecipitated using anti-EE-tag antibody. The immune complexes and total cell lysates were separated by SDS-PAGE and immunoblotted with pS6K(T412), anti-EE tag or anti-actin antibodies. The data shown in FIG. 4D clearly indicate that substitution of K99 to R does not effect T412 phosphorylation, while K104R mutant is defective in T412 phosphorylation. The level of T412 phosphorylation was standardized to the level of recombinant S6K1 in total lysates or immunoprecipitates. In addition, reprobing of membranes with anti-actin antibody indicated that equal amount of total protein was loaded from each sample.

### Example 5

The identification of several S6K acetylation sites allowed the study of site-specific acetylation using point mutants. It has been demonstrated that mutation of lysine to glutamine can mimic acetylation in a manner analogous to substitution of serines or threonines with acidic residues to mimic phosphorylation. The glutamine residue is neutrally charged at physiological pH and contains a carbonyl group at the end of the side chain, two characteristics possessed by acetylated lysine. Arginine is most commonly substituted for lysine to produce a non-acetylated mutant since, like lysine, it possesses a basic side chain but cannot be acetylated. All acetylation sites identified by mass spectrometry were mutated to arginine and to glutamine in order to study the possible effects of site-specific acetylation on S6K function. Since the P-loop is involved in ATP binding, we hypothesized that acetylation of the P-loop lysines may modulate the kinase activity of S6Ks in some way. In order to investigate this possibility, double mutants in which both P-loop lysines were replaced either with arginine or with glutamine were expressed in 293 cells and immunoprecipitated along with wild type S6Ks. Immune complex S6K assays were conducted and gels were exposed to a phosphorimager screen. These experiments were carried out on three separate occasions and in each case it was clear that mutation of these lysine residues has a significant inhibitory effect on the activity of S6Ks (FIGS. 4 A and B). While the activity of the arginine mutant is significantly lower than that of the wild type, mutation to glutamine almost completely abrogates the S6K activity. These data suggest that while it is critical to have lysine at these positions (even the relatively subtle change to arginine reduces activity), acetylation of the lysines (mimicked by the glutamine mutant) may be a way in which S6K activity can be down-regulated by acetyltransferases. Note that phosphorylation of the critical rapamycin-sensitive S6K activation site, threonine 412 is much reduced in the P-loop mutants (FIG. 4C).

To further elucidate the importance of individual lysine residues in the P loop, we have made mutants which have single K to R substitutions at positions K99 and K104 in S6K1. The wt S6K1 and generated mutants, K99R and K104R, were transiently expressed in Hek 293 cells and immunoprecipitated using anti-EE tag antibody. Total cell lysate and immune complexes were resolved by SDS-PAGE and immunoblotted with antibodies specific for pT412, EE-tag and actin. FIG. 4D demonstrate that the phosphorylation of K99R mutant is equal to that of wtS6K1 in total lysates and immune complexes. It important to state that the level of T412 phosphorylation in different samples is standardized to the level of actin or recombinant S6K1. In contrast to K99R mutant, the phosphorylation of K104R mutant at T412 was significantly inhibited. This is an intriguing observation, however it is not yet clear under what conditions these particular modifications may act to regulate S6K activity.

We are currently making K99Q and K104Q mutants, which should mimic the acetylation status of S6K1. The analysis of these mutants would allow us to further elucidate in vivo the role of acetylation of S6K in the P loop.

Immune complex kinase assays conducted using the S6K1 K516R/Q and the S6K2 K298R/Q mutants immunoprecipitated from asynchronous growing cells revealed no obvious differences from the wild type proteins (data not shown).

### Example 6

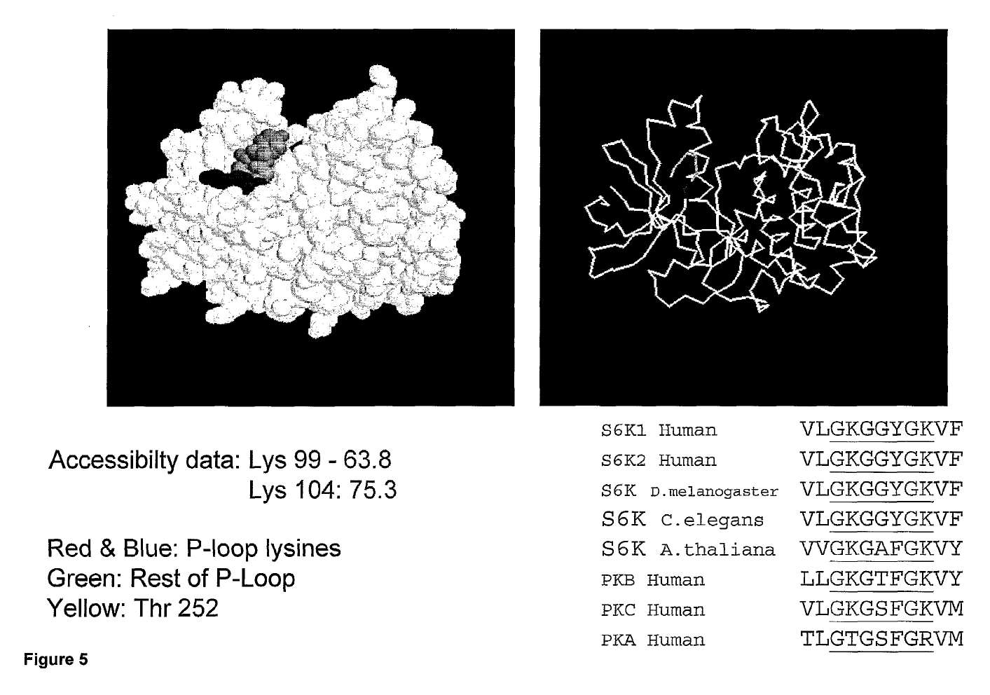

The model of S6 kinase domain indicates that P loop lysines are well accessible for posttranslational modifications. The 3D structure of S6K has not been solved so far. Therefore, it was decided to generate a model of S6 kinase domain, based on the crystal structure of PKB/Akt. A model was then generated using MODELLER7 (Sali & Blundell (1993) J. Mol. Biol. 234, 779-815) with standard comparative modelling parameters. Solvent accessibilities were then calculated using the method of Lee & Richards (1971) J. Mol. Biol. 55, 379-400 as implemented in NACCESS (Hubbard and Thornton, unpublished). In the model, the kinase domain adopts the bilobal architecture characteristic of other members of the protein kinase family (FIG. 5). The P loop is located in the cleft between N- the C-terminal lobes of the kinase domain. In the model, K99 and K104 are shown in blue and red respectively and the rest of the loop in green. Solvent accessibility analysis clearly indicates that both lysines are well accessible for posttranslational modifications, such as acetylation.

Interestingly, sequence analysis of the B-RAF gene associated with human cancers has identified numerous single site missense mutations, mostly within the kinase domain. Most of identified mutations were found in two regions: the glycine-rich P loop of the N lobe and the activation segment. Structure analysis of kinase domains from B-RAF and insulin receptor in their inactive state showed that the P loop and the activation segment form an array of hydrophobic interactions. These interactions lock the kinase in the inactive conformation, which is not appropriate for the phosphotransfer reaction. Therefore, any posttranslational modifications in the P loop might affect the interaction between P loop and the activation segment.

### Example 7

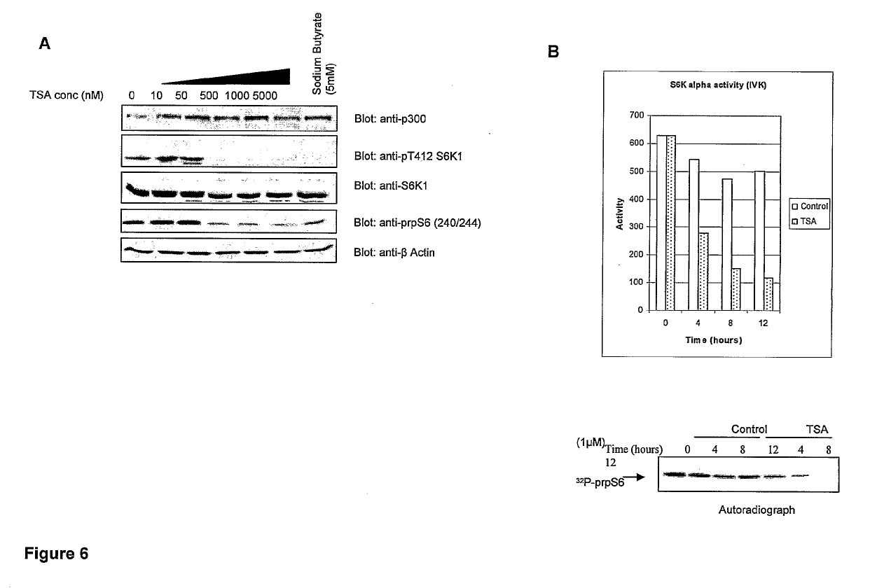

This example demonstrates that deacetylase inhibitors exhibit a strong inhibitory effect on S6K activity in a dose- and time-dependent manner. Since phosphorylation of multiple residues in S6 kinases has a potent effect on their kinase activity, it was interesting to see whether acetylation, an alternative post-translational modification, may also modulate the S6 kinase enzyme activity. Although kinase acetylation has not been described to date, there are examples of enzymes whose activity is regulated by p300-mediated acetylation, including Fen 1 (Hasan et al. (2001) Mol. Cell. 7, 1221-1231) and DNA polymerase beta (Hasan et al. (2002) Mol. Cell. 10, 1213-1222). In order to test whether S6 kinase acetylation may affect its kinase activity in vivo, C2C12 myoblasts were treated with a dose course of TSA. S6 kinase activation was analysed by western blotting with an antibody specific for phospho-threonine 412 and also with an antibody against the phosphorylated form of rpS6. FIG. 6A clearly shows that at doses of 500 nM TSA and above, S6 kinase activation is potently inhibited, as measured by both a reduction in phospho-threonine 412 and S6 protein phosphorylation. In order to further investigate this effect, time course experiments were conducted in which C2C12 cells were treated with 1 μM TSA in combination with 10 mM nicotinamide for various time periods prior to analysis of S6 kinase activity. In this case, S6K1 and 2 were immunoprecipitated from the cell lysates and subjected to immunocomplex kinase assays using purified ribosomes as substrate. Lysates were again blotted for pT412 and prpS6. FIG. 6B shows that treatment of C2C12 cells with a combination of the class I/II HDAC inhibitor, TSA and the SIRT1 deacetylase inhibitor, nicotinamide, causes a significant time-dependent reduction in S6 kinase activity as assessed by in vitro kinase assay and by western blotting with phospho-specific antibodies. Interestingly, the activation status of an upstream pathway member, PKB, as analysed by an antibody specific for phospho-Foxo3a (a well defined PKB substrate) appears to be unaffected by TSA/nicotinamide treatment (data not shown). This would suggest that TSA affects the S6 kinase pathway downstream of PKB thus reducing the likelihood that receptor tyrosine kinases, PI3K, PTEN, PDK-1, or indeed PKB itself, are the targets through which the TSA effect is mediated.

### Example 8

Histone deacetylase inhibitors downregulate S6K activity in a range of different cell types. In order to test the model that S6K acetylation may be playing a role in its de-activation, the effect of TSA on S6K activity was analysed in a number of cell types, some of which contain mutations in the pathways leading to S6K activation, thus affording a genetic analysis of this phenomenon. FIG. 6A shows that S6K activity was inhibited (to a greater or lesser extent) by deacetylase inhibition in all cell types tested. Interestingly, the effect was retained in PC-3 cells. PC-3 is a PTEN-null prostate cancer cell line in which basal PKB and S6K activation is high due to deregulation of PI3K signalling. The fact that S6K is inhibited by TSA treatment of these cells suggests that this effect is exerted downstream of PTEN, i.e. the inhibitors do not act by affecting a receptor tyrosine kinase or PI(3)K. Furthermore, S6K activity is affected by deacetylase treatment of MKOC cells (FIG. 6B). The MKOC cell line was derived from a renal tumour removed from a Tsc2^{-/-} mouse. Since these cells lack Tsc2, S6K activity is uncoupled from both PKB and the tuberous sclerosis complex heterodimer. The fact that TSA causes reduction of S6K activity in these cells suggests that mTOR or S6K itself may well be the point at which deacetylase inhibition is acting. Blotting with an antibody against phospho-4EBP1, the other major target of mTOR revealed that its phosphorylation status is unaffected by TSA treatment, suggesting that S6K itself and not mTOR, is affected by deacetylase inhibition (FIG. 6B). It should be noted that, although the same trend (reduced S6K phosphorylation and activity following treatment with deacetylase inhibitors) is seen in all the cell lines tested, the magnitude of this effect does vary. The relatively minor decrease in rpS6 phosphorylation status in MCF-7 cells and MKOC cells may be indicative of another functional, ‘deacetylase inhibitor-insensitive’ S6 kinase, in these cells, possibly S6K2. Indeed, MCF-7 cells express high levels of S6K2. Significantly, phosphorylation of S6K1 on T412 and S6K1 activity as measured by immune complex kinase assay is reduced in MKOC cells upon treatment with HDAC inhibitors (FIG. 6B).

These observations suggest two obvious explanations. Firstly, that TSA is not a specific inhibitor of HDAC activity but that it is also a direct S6K inhibitor in the classical sense. The second explanation is that through inhibiting HDACs, TSA causes accumulation of an acetylated species of S6K that is catalytically inactive. In order to test the former explanation, that TSA is a direct S6K inhibitor, in vitro kinase assays were conducted using recombinant purified S6K in the presence or absence of TSA and nicotinamide (FIG. 6C). This experiment does not indicate that TSA inhibits S6K activity in vitro and so is unlikely to be acting as a direct kinase inhibitor.

### Example 9

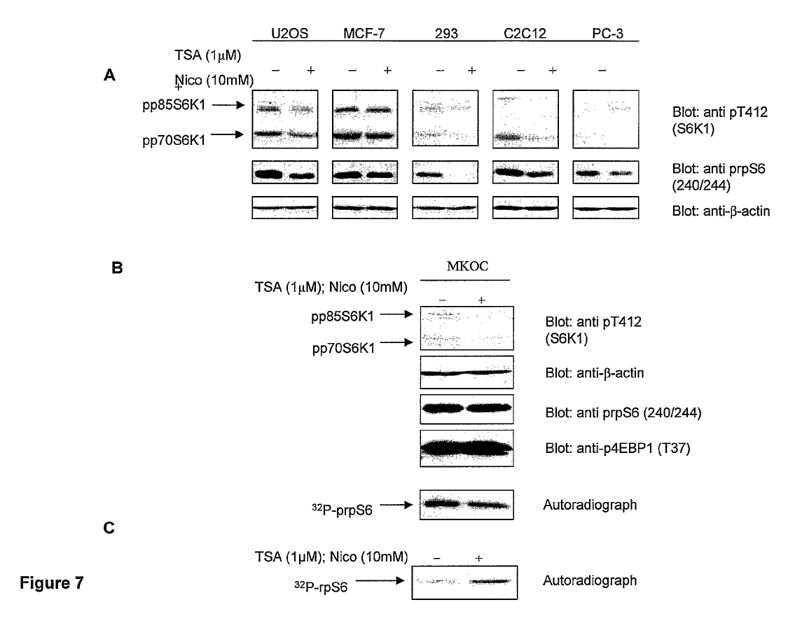

Analysis of endogenous S6K1 acetylation during the cell cycle. In order to learn more about the potential role of S6K acetylation, we sought to determine the physiological conditions under which it becomes acetylated. Since stimulation of quiescent cells induces complex formation between S6K1 and p300, it was pertinent to establish whether this event results in S6K1 acetylation. NIH-3T3 cells were used for this analysis, since, in our experience, they are amenable to synchronisation and express relatively high levels of S6K1. For synchronisation, the cells were grown to 50% confluence and the medium was replaced with DMEM containing 0.5% serum for a starvation period of 24 hours. Cells were then stimulated by re-addition of DMEM/10% serum for various time periods. Time points were taken up to 24 hours in order to analyse how S6K1 acetylation may be regulated throughout the cell cycle. S6K1 was immunoprecipitated from lysates using a monoclonal antibody and immune complexes were washed extensively prior to SDS-PAGE. Acetylated S6K1 was detected by western blotting with anti-acetyl lysine antibodies as described in chapter four. Blots were then stripped and reprobed with polyclonal antibodies against S6K1. FIG. 6 shows that in the absence of serum, no S6K1 acetylation was detected. Note that no interaction between S6Ks and acetyltransferases p300 or P/CAF was detected in starved MCF-7 cells (FIG. 1A). S6K1 acetylation is clearly detected one hour post-stimulation and the acetylation signal appears to increase towards the later stages of the cell cycle, particularly between 17 hours and 21 hours. This correlates with the end of S-phase and the passage through G₂ to M-phase, as determined by FACS analysis of these cells (data not shown). Interestingly, speckled nuclear staining of S6K1 has been reported during late S-phase in Swiss-3T3 cells, suggesting a possible association with chromatin at this time. The lysates from these cells were subjected to western blotting with antibodies against phospho-T412 (S6K) and phospho-rpS6, to assess the S6K activity at each time point. The profile obtained (FIG. 7, lower panels) matches that seen for S6K activity throughout the cell cycle of a related mouse fibroblast cell line, Swiss 3T3: S6K1 pT412 phosphorylation and rpS6 phosphorylation peak 30 minutes to one hour following serum stimulation and gradually fall to around 40% of the maximal activity. It appears then, that S6K acetylation peaks at a time when phosphorylation and activity are low.

### Example 10

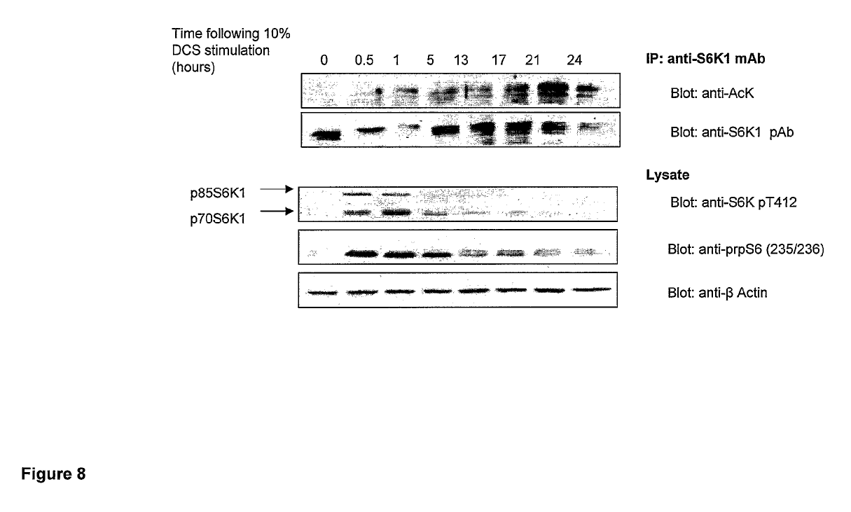

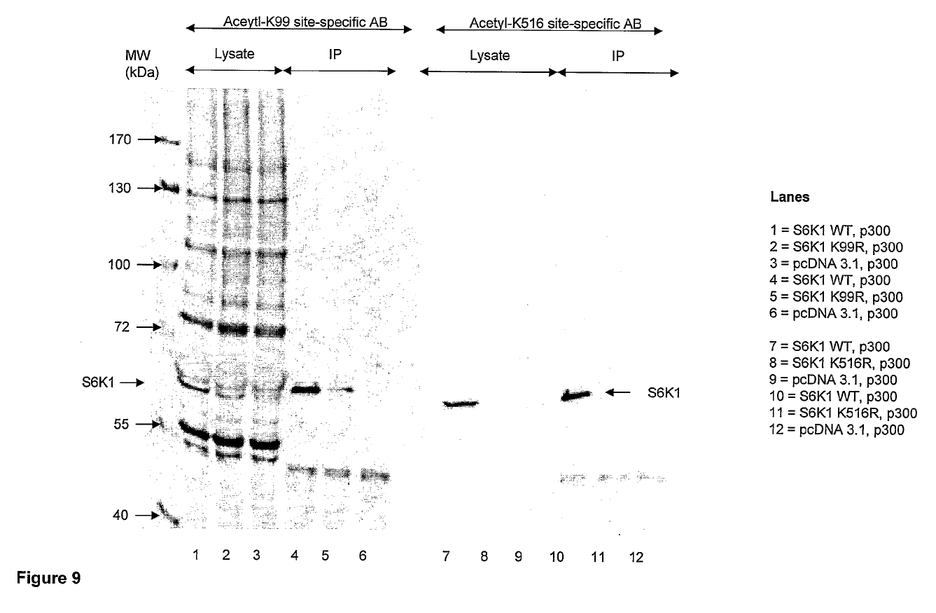

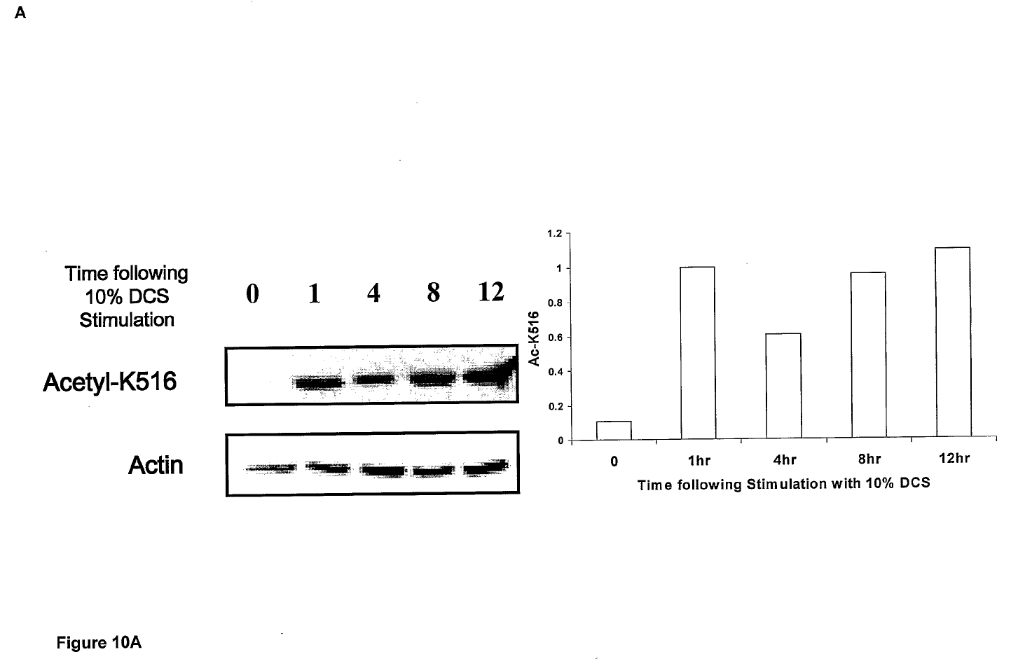

Testing the specificities of antibodies directed against identified acetylated lysines. Acetyl-lysine specific antibodies are a powerful tool in investigating the physiological importance of protein acetylations. A number of highly specific acetyl-lysine site-directed antibodies have been raised and used successfully to study the physiological relevance of these modifications in histones, transcription factors and signaling molecules. We therefore attempted the production of a panel of antibodies that would specifically recognize acetylated forms of S6K1 at K99, K104 and K516. The antibodies were raised in rabbits by injecting short synthetic peptides containing acetylated lysines in the middle of the peptide chain. Produced antibodies were affinity purified in two steps. Initially, Affigel beads coupled with antigenic peptides were used as matrices for purifying generated antibodies. Then, in order to remove IgGs which are directed to non-acetylated peptides, the antibodies obtained in the first step of purification were loaded onto the column containing corresponding non-acetylated peptides. The non-bound fractions were used for testing the specificity of generated antibodies. We have purified very small amount of antibodies against Ac-K104 peptide, so they were not included in the specificity test. The wtS6K1 or corresponding mutants (K99R and K516R) were transiently overexpressed in Hek293 cells. Cells transfected with vector alone were used as a negative control. Total cell lysates or immunoprecipitated recombinant S6Ks were resolved by SDS-PAGE and probed with Ac-K99 or Ac-K516 antibodies. As shown in FIG. 8, affinity purified Ac-K516 antibodies specifically recognized wtS6K1 in immune complexes and in total lysates, but not the mutant K99R form of the kinase. These data clearly indicate that generated antibodies are highly specific towards the acetylated S6K1 at K516 and could be used on total cell and tissue lysates. As shown in FIG. 9, Ac-K99 antibodies have to be further purified, since they recognize not only wtS6K, but also K99R mutant (to a lesser extent).

### Example 11

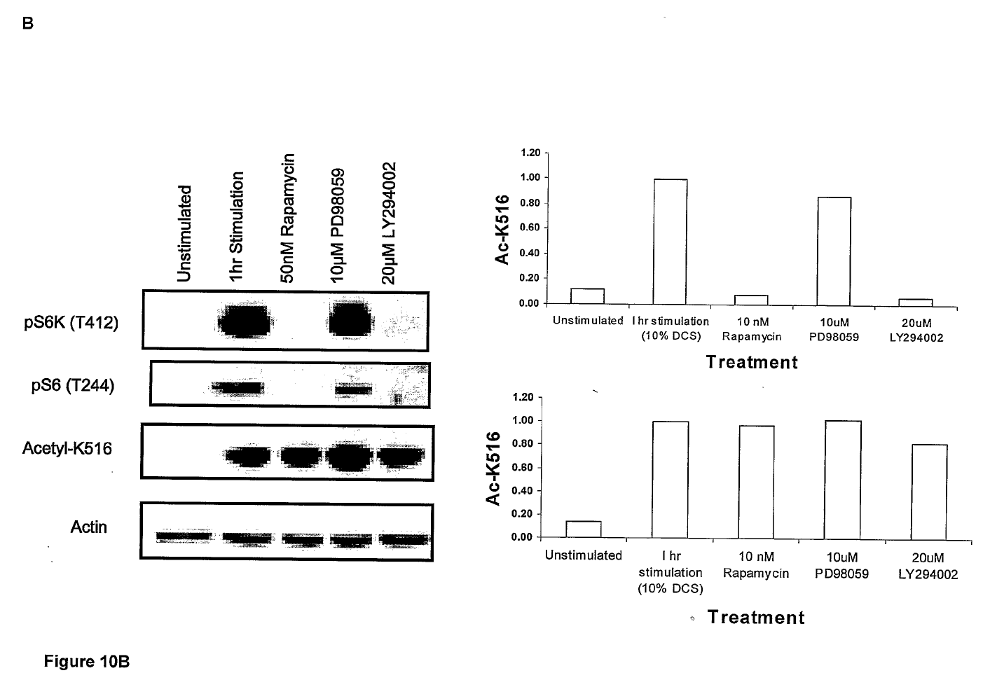

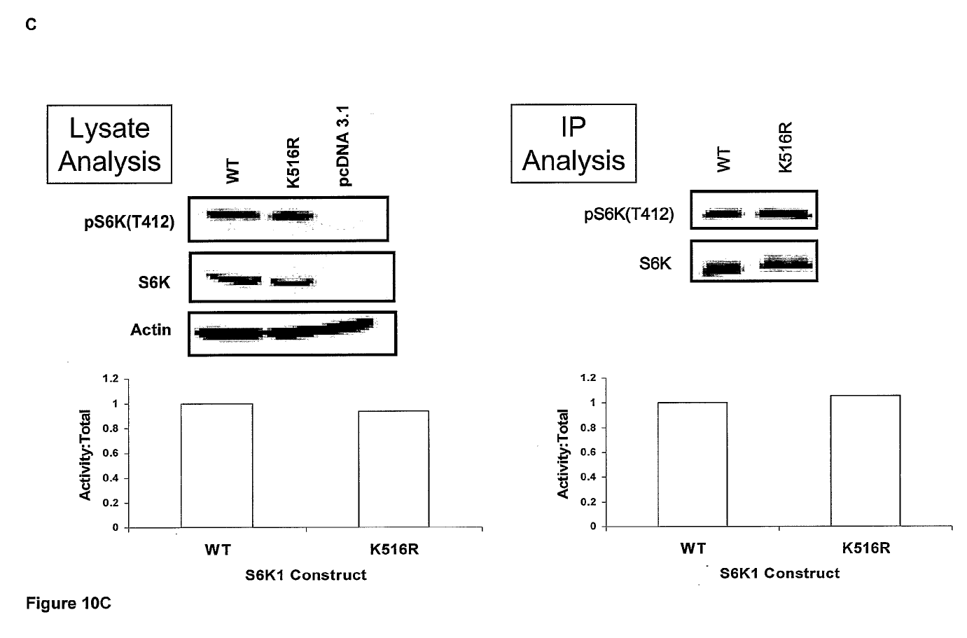

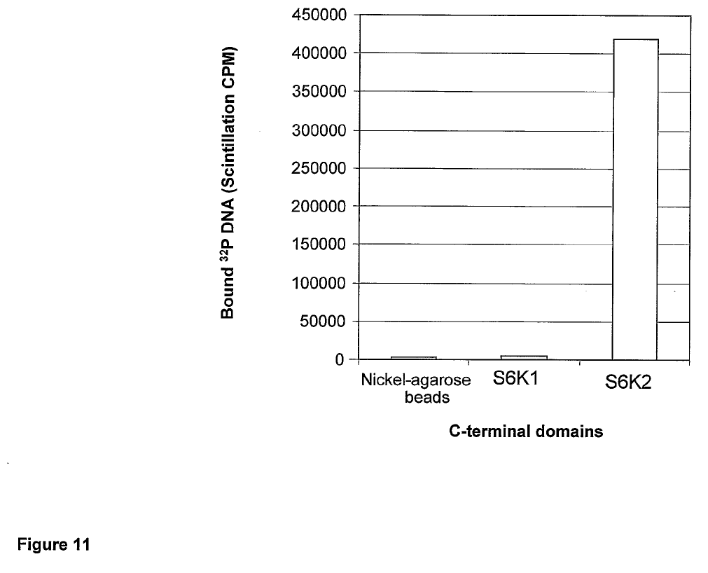

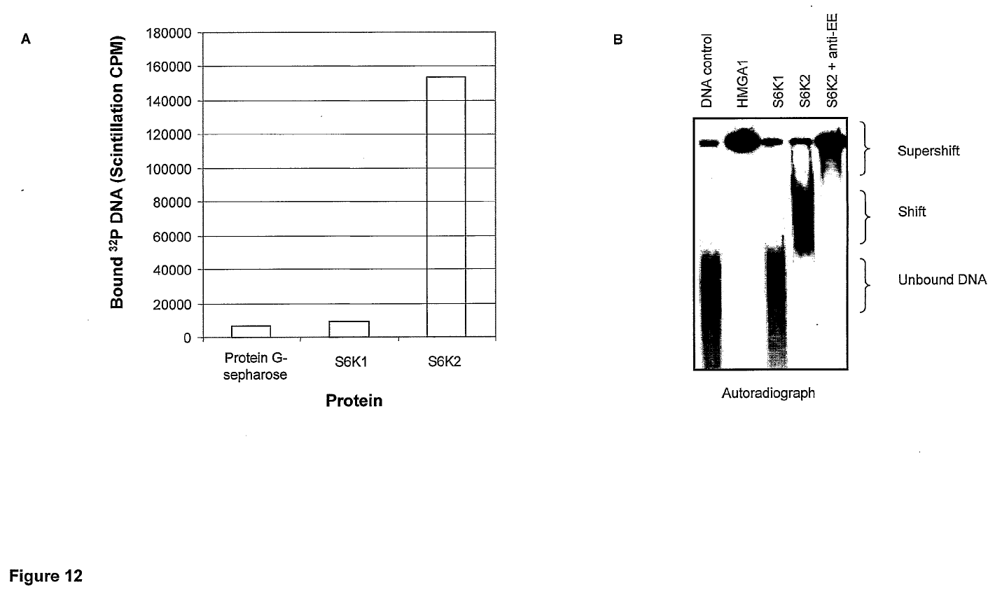

Endogenous S6K1 is acetylated at K516 in serum-stimulated cells. The availability of highly specific Ac-K516 antibodies has allowed us to study the acetylation status of S6K1 at K516 in response to mitogenic stimuli and in cells treated with inhibitors of major signal transduction pathways. Initially, acetylation of K516 in starved and serum stimulated NIH 3T3 cells was tested. In this study, NIH 3T3 cells were starved for 24 hrs and stimulated with serum for various period of time. Total cell lysates were separated by SDS-PAGE and immunoblotted with anti-actin and Ac-K516 antibodies. The results presented in FIG. 10A clearly indicate that acetylation at K516 does not occur in starved cells, but is strongly observed after serum stimulation.

Furthermore, the effect of signal transduction inhibitors on K516 acetylation was studied in NIH 3T3 cell. In this experiment, cells were serum starved and treated with rapamycin, LY and PD for 30 minutes before serum stimulation. Cells were lysed 1 hr hour after stimulation and the lysates separated by SDS-PAGE and immunoblotted with Ac-K516, pT412 antibodies. As shown in FIG. 10B, acetylation of S6K1 at K516 was induced by serum stimulation, but not affected by any of signal transduction inhibitors used in this study. It is important to note that Rapamycin and LY294002 strongly inhibited phosphorylation of S6K1 at T412 and ribosomal protein S6 at T244. These data suggest that acetylation of S6K1 at K516 does not affect its kinase activity.

Mutational analysis of this site further supported this hypothesis. In this study, wtS6K1 and K516R mutant were expressed in Hek293 cells. Cells transfected with vector alone were used as a control. Transiently expressed S6Ks were immunoprecipitated with anti-EE tag antibodies and the immune complexes together with the total cell lysates were separated by SDS-PAGE. The Western blot analysis showed no significant changes in the level of T412 phosphorylation between the wild type and mutant form of S6K1 (FIG. 10C). The level of T412 signal was standardized to the level of actin and recombinant S6K in each sample.

### Example 12

S6K2 possesses a putative AT-hook DNA Binding Motif. Screening of the NCBI protein sequence databases with short segments of the S6K2 revealed the presence of a sequence with high homology to a DNA binding motif known as the AT hook. This motif is found at the extreme C-terminus of S6K2 (residues 487 to 495), adjacent to the nuclear localization sequence. The AT hook has been described in only a few proteins, the canonical example being the high mobility group protein HMG1A (formerly HMGI(Y) (reviewed in Reeves (2001) Gene 277, 63-81). The AT hook is a short DNA binding motif that binds to the minor groove of short (4 to 6 base pairs) ‘AT-rich’ DNA.

Table 2 shows the alignment of AT hook sequences from several cellular proteins, including S6K2. The AT hook is a non-classical DNA binding motif found in a growing number of proteins with a variety of functions, including transcription factors and other transcriptional regulators, protein kinases and phosphatases. AT hook-containing proteins are known to play a number of roles, including the repression or potentiation of specific gene transcription via binding to gene promoter/enhancer elements (Reeves et al. (2001). Among the several reported roles for HMGA1 is participation in the enhanceosome, which controls expression of the interferon-β gene (Thanos and Maniatis (1995) Cell 83, 1101-1111). Another example is the recently described AKNA transcription factor, which controls the expression of CD40 and CD40 ligand in B-lymphocytes (Siddiqa et al. (2001) Nature 410, 383-387). Other functions include the initiation of DNA replication in yeast via binding at origins of replication, maintenance of heterochromatin and signal transduction to the nucleus.

TABLE 2
Alignment of A-T hook sequences

| | AT-hook | |
|---|---|---|
| Protein | sequence | Protein function |
| AT-hook consensus | KRKRGRPRK | |
| S6K2 | KRGRGRPGR | Ser/Thr kinase |
| HMGA | KRGRGRPRG | Transcription |
| | | factor |
| D-1 | -KGRGRPKK | Unclear |
| AKNA | -RTRGRPAD | Transcription |
| | | factor |
| HP2 | KRKRGRPRK | Gene silencing |
| Orp4p | KRGRGRPRL | S. pombe DNA |
| | | replication |
| DNA-PK | KRVRGRPCL | DNA repair |
| VRK1 | KKSRGRPKK | Ser/Thr kinase |
| PSK-C3 | KRRRGRPPK | Phosphorylase |
| | | kinase |
| Methyltransferase 8 | KRGRGRPKG | Histone H3 methyla- |
| | | tion |
| MeCP-2 protein | GRGRGRPKG | Methyl-CpG-binding |
| | | protein |
| Zinc finger prot 278 | KRGRGRPRK | Transcription |
| | | factor |

### Example 13

The AT-hook motif is evolutionary conserved in S6K2. To gain the insight into functional importance of S6K2 AT-hook motif, it was examined to see whether the motif is conserved in evolution. The alignment of the S6K2 C-terminal protein sequences from various species clearly indicated that the AT-hook motif is highly conserved (see Table 3). The central RGRP motif is flanked with characteristic basic residues, which contributes to DNA binding. Three AT-hook subtypes have been described, all bearing the central RGRP motif. The S6K2 sequence is very similar to the type II AT-hook consensus.

TABLE 3

Evolutionary conservation of the
AT hook motif in S6K2

| | |
|---|---|
| Human S6K2 | KRGRGRPGR |
| Rat S6K2 | KKGRGRPGR |
| Bovine S6K2 | KRGRGRPGR |
| Pig S6K2 | KRGRGRPGR |
| Dog S6K2 | KRGRGRPGR |

### Example 14

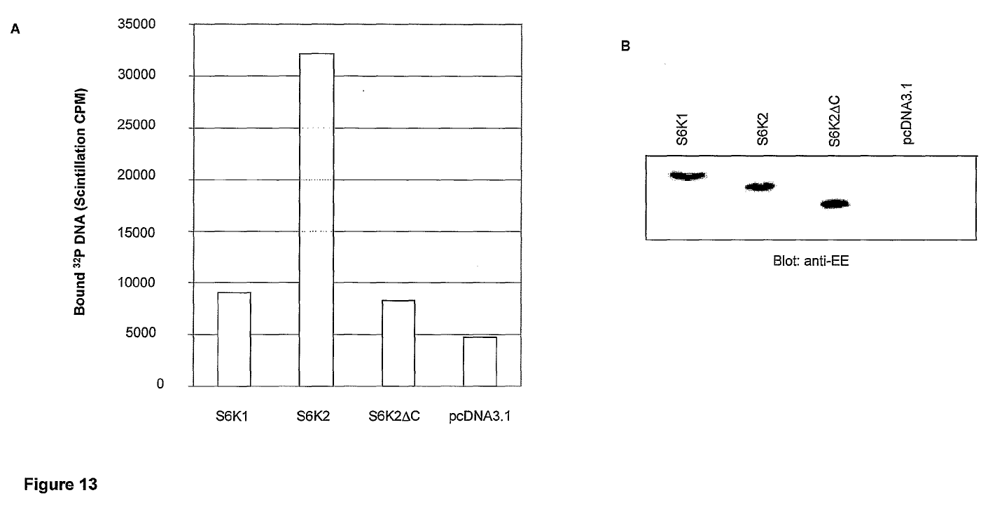

The C-terminal domain of S6K2 possesses DNA binding ability. Since a putative DNA binding motif was identified in the S6K2 C-terminal domain, it was pertinent to establish whether this motif may function in a simple biochemical assay. Two approaches were adopted in order to assess the DNA binding activity of S6K2: an on-beads DNA binding assay in which the DNA binding activity of immobilised recombinant protein to radiolabelled DNA is measured by scintillation counting and also an electrophoretic mobility shift assay (EMSA), in which binding of recombinant protein to DNA was measured by retardation of the DNA mobility in a non-denaturing gel, relative to free DNA control. Since the putative DNA binding activity is found at the S6K2 C-terminus, initial experiments were carried out using recombinant 6×His-tagged S6K2 C-terminal domain purified after expression in bacteria (hereafter referred to as S6K2CT). The C-terminal domain of S6K1 (S6K1 CT) was also included in these experiments as a negative control. It can be seen from FIG. 13 that the S6K2 C-terminal domain binds to DNA in vitro and that background binding of DNA to nickel-NTA agarose beads or to the S6K1 C-terminus was very low by comparison.

### Example 15

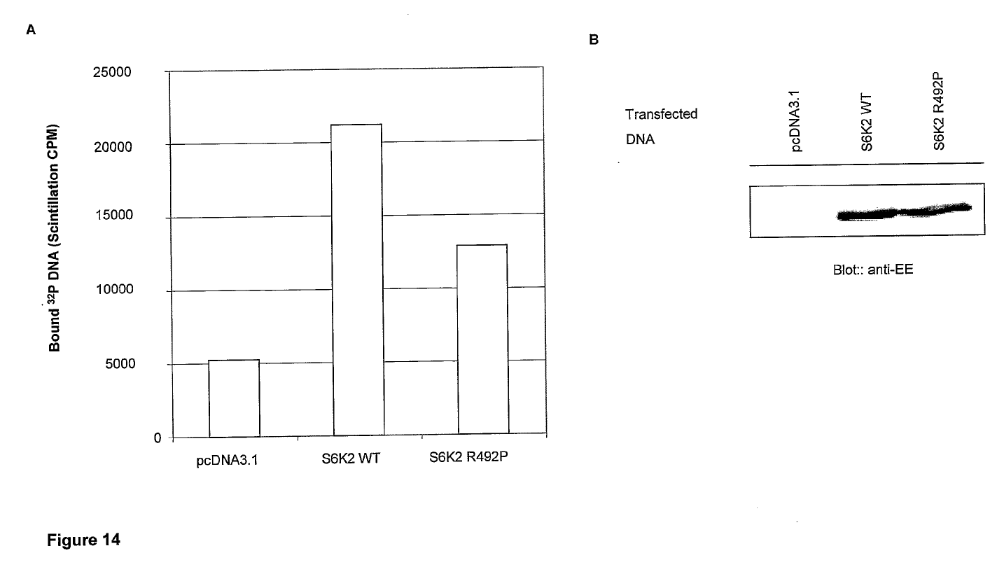

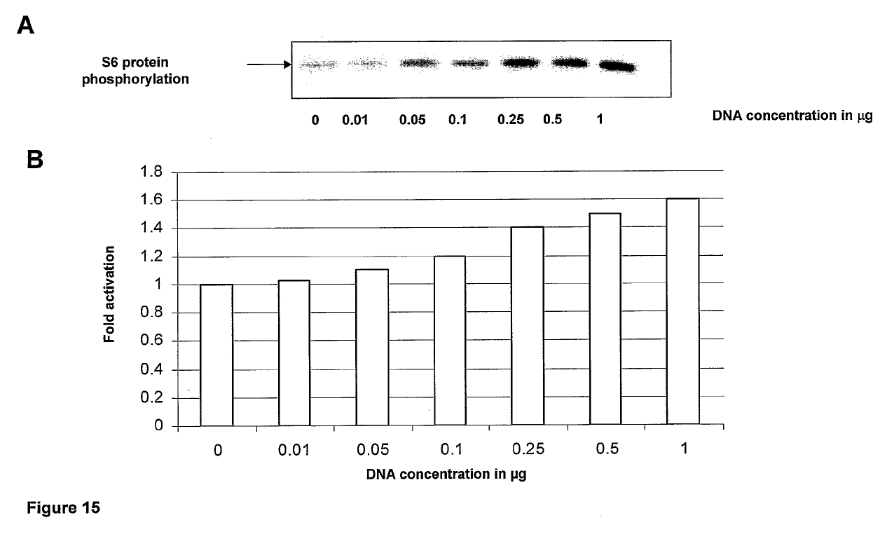

The full-length S6K2 but not S6K1 binds to DNA in vitro. Having established that the C-terminal domain of S6K2 can bind directly to DNA in vitro, it was next investigated whether this DNA binding activity is retained in the context of the full-length protein. Purified EE-tagged S6Ks derived from baculoviral expression in sf9 cells were assayed for their DNA binding activity using the methods described for the C-terminal domains earlier. Once again, S6K1 was used as a negative control. FIG. 15A shows that the DNA binding activity displayed by S6K2CT appears to be preserved in the full length protein, while full length S6K1 appears unable to bind DNA. Furthermore, the DNA binding activity of S6K2 was able to be demonstrated using the EMSA approach. FIG. 14 clearly shows a shift in the DNA when it is pre-incubated with S6K2 relative to the free DNA control (compare lanes 1 and 4). This indicates that a protein-DNA complex is formed, thus increasing the size and decreasing the mobility of the labeled DNA. The specificity of this interaction is indicated by both the lack of a mobility shift when DNA is incubated with S6K1 (lane 3) and by the ‘supershift’ produced by addition of the anti-EE antibody to the DNA/S6K2 mixture (lane 5). In this case, the antibody binds to the epitope tag on S6K2, thus forming a tertiary complex that further decreases the mobility. As discussed earlier, this technique is more commonly applied to EMSA experiments in which a mixture of proteins (typically a nuclear extract or total cell lysate) is incubated with a specific DNA probe. In this case, a number of proteins in the mixture may be expected to bind the DNA, so a shift cannot be attributed to a particular protein. Here, addition of an antibody specific to the protein of interest is used to produce a supershift, indicating binding of the protein of interest to the DNA probe.

FIG. 14B also shows binding of the positive control AT-hook protein, HMG1A to DNA. In this case, the protein-DNA complexes failed to enter the gel and remained in the well. This is effect may be overcome by reducing the amount of protein.

### Example 16

The deletion of the S6K2 C-terminal domain abolishes DNA binding. The experiment described in FIG. 15 demonstrates that the S6K2 C-terminal domain is sufficient for DNA binding but it still needed to be determined whether this domain is required for DNA binding of the full-length protein. In order to investigate whether this is indeed the case, or whether there may be other DNA binding regions present in full length S6K2, a truncation mutant of S6K2 lacking the C-terminal domain (S6K2ΔCT) was assayed for its DNA binding activity. Truncation mutants of S6K1 and S6K2, lacking regulatory N- or C-terminus were made. These truncated mutants were fused with the EE tag epitope sequence at the N-terminus. EE-S6K1, EE-S6K2 or EE-S6K2ΔCT were expressed in HEK 293 cells by transient transfection and immunoprecipitated the recombinant proteins using anti-EE antibodies. The immunocomplexes were washed under stringent conditions before incubation with labeled DNA as described earlier. FIG. 15A shows that the DNA binding activity of S6K2ΔCT is significantly lower than that of the full-length protein, suggesting that the DNA binding activity of S6K2 is indeed conferred by the C-terminal domain. Equal amounts of the wild type and S6K2ΔCT were used in the experiment as determined by western blotting of the cell lysates with EE monoclonal antibodies (FIG. 15B) Note that even with the stringent washing conditions employed, it could be argued that full length S6K2 co-precipitates with a second DNA-binding protein that is not able to bind S6K2ΔCT. The fact that in vitro binding of highly purified S6K2 has been demonstrated would argue against this conclusion.

### Example 17

Substitution of a conservative residue within S6K2 AT-hook core motif inhibits DNA binding. Having demonstrated that the DNA binding activity of S6K2 is contained within the C-terminal domain of the protein, it was interesting to establish whether this activity is due to the putative AT-hook identified by sequence homology. In order to test this hypothesis, it was decided to disrupt the S6K2 AT-hook by site directed mutagenesis of a residue highly conserved in all known AT-hooks. The critical determinant residues for AT-hook function are the Arg-Gly-Arg-Pro sequence. Binding of this motif to the minor groove of AT rich sequences has been studied in detail and the interactions between these residues and the DNA are known (Reeves and Nissen (1990) Biol. Chem. 265, 8573-8582). The N—H group on the amide bonds between the glycine and the second arginine residue in this core motif forms a hydrogen bond with the DNA phosphate backbone. In the absence of any published data on mutational analysis of AT-hook motifs, it was reasoned that replacement of this arginine with proline would disrupt the overall structure of this motif and also remove the N—H group from the amide bond. Site directed mutagenesis was carried out on the full-length S6K2 sequence in pcDNA3.1. The substitution of Arg 492 to Pro was confirmed by DNA sequencing. The mutant cDNA (termed S6K2 R492P) was transfected into HEK 293 cells in parallel with S6K2 wild type. The proteins were immunoprecipitated and subjected to DNA binding assays. FIG. 15B shows that mutation of Arg 492 to proline results in a considerable loss of DNA binding activity. Taken together, these data suggest that S6K2 does indeed possess a functional AT-hook motif that bestows a non-specific DNA binding activity on the protein. Loss of the region encoding this motif, or direct disruption of the motif by site directed mutagenesis abrogates the DNA binding activity of S6K2.

### Example 18

The binding of DNA to S6K2 stimulates its kinase activity. The regulatory N and C-terminal regions of S6K1 and S6K2 are known to regulate their kinase activities. Interaction between regulatory N-terminal acidic and C-terminal basic domains is thought to maintain S6K1 in an inactive conformation in unstimulated cells. In this inactive state, a pseudosubstrate region in the C terminus may hinder the catalytic site. Phosphorylation of the C-terminal proline-directed motifs is thought to disrupt the autoinhibitory interaction of the N- and C-terminal domains, exposing other critical regulatory sites in the linker and catalytic regions. It has been shown that deletion of the C-terminal domain of S6K2 enhances kinase activity whereas analogous deletion of S6K1 is inhibitory. The authors also found that the inhibitory effects of the S6K2 C-terminal domain are partly attributable to the nuclear localization signal and to the C-terminal proline-directed phosphorylation sites. The nuclear localization signal overlaps with the AT-hook DNA binding motif. Considering this, it could predicted that binding of DNA to the basic region of S6K2 C-terminal domain which possesses NLS and an AT-hook motif may affect the autoinhibitory interaction of the N- and C-terminal regions. To test this hypothesis, EE-tagged forms of S6K1 and S6K2 were expressed in insect cells using recombinant baculoviruses. Both kinases were purified nearly to homogeneity using affinity matrixes with EE monoclonal antibodies. Purified preparations of S6K1 and S6K2 were used in in vitro kinase in the presence or absence of sonicated genomic DNA. Ribosomal S6 protein was used as a substrate in the kinase assay. The level of S6 phosphorylation was measured by SDS-PAGE and phosphorimager. The level of S6 phosphorylation by S6K2 was found to be reproducibly higher in the presence of DNA. The presence of absence of DNA did not affect the activity of S6K1 towards ribosomal S6 protein. Dose-course stimulation of S6K2 activity by DNA was carried out. The S6 kinase reaction was carried out in the presence of various concentrations of DNA. In several independent experiments, a dose-dependent activation of S6K2 activity by DNA was found (FIG. 15B). A 1.5 to 2 fold increase of S6K2 activity in the presence of 1 mg of DNA. It is an interesting observation, since only few protein kinases are known to possess DNA-binding potential and more importantly to be activated by interaction with DNA. The best example is the DNA dependent protein kinase (DNA-PK) (Lees-Miller et al. (1990) Mol. Cell. Biol. 10, 6472-6481). Using a nuclear fractionation protocol, a small fraction of S6K2 in the nuclear matrix fraction was detected. This is consistent with an earlier report describing nuclear S6K2 isoform that is insoluble in standard detergents (Lee-Fruman et al. (1999) Oncogene 18, 5108-5114). Digestion of this fraction with DNase1 appeared to solubilize a portion of S6K2, providing an indication that is indeed associated with chromatin in cells. Since scaffold attachment, regions are known to be sites of transcriptional activity, containing highly acetylated histones (Davie (1997) Mol. Biol. Rep. 24, 197-207), localization of S6K2 to these regions would be consistent with a role in transcriptional regulation.

Based on the examples, it was found for the first time that S6K2, but not S6K1 possesses an AT-hook DNA binding motif. Furthermore, it was demonstrated that S6K2 AT-hook motif mediates specific association with DNA. The consequence of S6K2-DNA interaction is the induction of its kinase activity. This is the first time the presence of a functional AT-hook DNA binding motif in the family of protein kinases has been described. Furthermore, these findings provide new insight into the mechanism of S6K2 signaling in the nucleus. Based on these data, a novel mechanism by which S6K2 can transduce growth-promoting effects in response to mitogenes and nutrients is proposed. This may involve the regulation of transcription factors and/or chromatin remodeling proteins by phosphorylation, when S6K2 complexes with DNA and is activated by this interaction. It is important to note that this discovery has an important implication for the development of drugs, which can block S6K2-DNA interaction and subsequently its growth promoting functions, which are deregulated in cancer and diabetes.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

1. An isolated peptide or derivative thereof comprising a binding domain of p300 acetyltransferase of less than 200 amino acids of SEQ ID NO: 4 wherein the binding domain is capable of binding to and modulating an S6 kinase protein activity.

2-4. (canceled)

5. The isolated peptide or derivative thereof of claim 4, wherein the site of acetylation is at an amino acid corresponding to residue 516 of an S6 kinase protein.

6. The isolated peptide or derivative thereof of claim 5, wherein said residue is lysine.

7. The isolated peptide or derivative thereof of claim 1, wherein the isolated peptide comprises the amino acid sequence of SEQ ID NO: 6 (p300-4).

8. (canceled)

9. An isolated nucleic acid molecule encoding the isolated peptide or derivative thereof of claim 1.

10. (canceled)

11. A method of identifying an agent which binds to and/or modulates the acetylation of the S6 kinase protein, comprising:
(a) exposing the S6 kinase protein to the agent in the presence of the isolated peptide of claim 1, and
(b) measuring the specific binding of the isolated peptide to the S6 kinase protein and/or measuring the acetylation of the S6 kinase protein by the isolated peptide, wherein a decrease in the binding of the isolated peptide and/or a change in acetylation of S6 kinase compared to a control is indicative of an agent capable of binding to the S6 kinase protein.

12. (canceled)

13. The method of claim 11 further comprising contacting the S6 kinase protein with the isolated peptide of claim 1 prior to measuring acetylation.

14. The method of claim 11 wherein the acetylation is at a lysine residue at position 516 of the S6 kinase protein.

15. The method of claim 14 wherein the activity of the S6 kinase protein is determined by measuring phosphorylation of one or more additional proteins.

16-20. (canceled)

21. A method of modulating an S6 kinase protein activity, comprising contacting the S6 kinase protein with the isolated peptide or derivative of claim 1.

22. The method of claim 21, wherein the S6 kinase protein activity is inhibited.

23. A method of treating a protein kinase related disease in a patient in need of such treatment comprising administering a therapeutically effective amount of a pharmaceutical composition comprising the isolated peptide of claim 1 capable of acetylating the protein kinase.

24-27. (canceled)

28. The method of claim 23, wherein said protein kinase related disease is selected from the group consisting of cancer, blood vessel proliferative disorders, autoimmune disorders, and metabolic diseases.

29. The method of claim 28 wherein said cancer is selected from the group consisting of squamous cell carcinoma, astrocytoma, Kaposi's sarcoma, glioblastoma, multiple myeloma, lung cancer, bladder cancer, head and neck cancer, melanoma, ovarian cancer, prostate cancer, breast cancer, small-cell lung cancer, glioma, colorectal cancer, genitourinary cancer, gastrointestinal cancer.

30. The method of claim 28 wherein said blood vessel proliferative disorder is selected from the group consisting of diabetic retinopathy, age-related macular degeneration, retinopathy of prematurity, arthritis and restenosis.

31. The method of claim 28, wherein said autoimmune disorder is selected from the group consisting of lupus erythematosus, discoid lupus erythematosus, subacute cutaneous lupus erythematosus, drug-induced lupus erythematosus, and systemic lupus erythematosus.

32. The method of claim 28, wherein said metabolic disorder is selected from the group consisting of psoriasis, diabetes mellitus, wound healing, inflammation and neurodegenerative diseases.

33. An antibody which specifically binds to an acetylation site on a kinase protein and is capable of inhibiting acetylation of the kinase protein by an acetyltransferase protein.

34. (canceled)

35. The antibody of claim 33 wherein the S6 kinase comprises the amino acid sequence of SEQ ID NO: 2.

36. The antibody of claim 33 wherein the antibody binds to a region in the S6 kinase comprising amino acid residue 516 of SEQ ID NO: 2.

37. The antibody of claim 34 wherein the amino acid residue is a lysine.

38. A method of identifying an agent that modulates the binding of S6 kinase 2 (“S6K2”) protein to a nucleic acid comprising:
(a) exposing the S6 kinase 2 protein to the agent; and
(b) detecting the binding of the S6 kinase 2 protein to the nucleic acid, wherein change in the level of binding compared to a control is indicative of an agent capable of modulating S6 kinase 2 binding to the nucleic acid.

39. The method of claim 38, wherein the binding to the nucleic acid is inhibited.

40. The method of claim 38 further comprising measuring of the activity of S6 kinase 2 protein.

41-50. (canceled)

51. The method according to claim 38, wherein the S6 kinase 2 protein is human S6 kinase 2 (SEQ ID NO: 8).

52-54. (canceled)

55. An isolated protein comprising one or more mutations at any amino acid residue corresponding to amino acids 487 to 495 of SEQ ID NO: 8.

56-60. (canceled)

61. A nucleic acid molecule encoding the isolated protein of claim 55 or a fragment thereof.

62-67. (canceled)


SEQUENCE LISTING (29 sequences)

SEQ ID NO: 1 - DNA, 2346 bp, Homo sapiens
  CDS (28)..(1602): p70(alpha) S6 Kinase gene
gcacgaggctgcggcgggtccgggcccatgaggcgacgaaggaggcgggacggcttttacccagccccggacttccgagacagggaagctgaggacatggcaggagtgtttgacatagacctggaccagccagaggacgcgggctctgaggatgagctggaggaggggggtcagttaaatgaaagcatggaccatgggggagttggaccatatgaacttggcatggaacattgtgagaaatttgaaatctcagaaactagtgtgaacagagggccagaaaaaatcagaccagaatgttttgagctacttcgggtacttggtaaagggggctatggaaaggtttttcaagtacgaaaagtaacaggagcaaatactgggaaaatatttgccatgaaggtgcttaaaaaggcaatgatagtaagaaatgctaaagatacagctcatacaaaagcagaacggaatattctggaggaagtaaagcatcccttcatcgtggatttaatttatgcctttcagactggtggaaaactctacctcatccttgagtatctcagtggaggagaactatttatgcagttagaaagagagggaatatttatggaagacactgcctgcttttacttggcagaaatctccatggctttggggcatttacatcaaaaggggatcatctacagagacctgaagccggagaatatcatgcttaatcaccaaggtcatgtgaaactaacagactttggactatgcaaagaatctattcatgatggaacagtcacacacacattttgtggaacaatagaatacatggcccctgaaatcttgatgagaagtggccacaatcgtgctgtggattggtggagtttgggagcattaatgtatgacatgctgactggagcacccccattcactggggagaatagaaagaaaacaattgacaaaatcctcaaatgtaaactcaatttgcctccctacctcacacaagaagccagagatctgcttaaaaagctgctgaaaagaaatgctgcttctcgtctgggagctggtcctggggacgctggagaagttcaagctcatccattctttagacacattaactgggaagaacttctggctcgaaaggtggagcccccctttaaacctctgttgcaatctgaagaggatgtaagtcagtttgattccaagtttacacgtcagacacctgtcgacagcccagatgactcaactctcagtgaaagtgccaatcaggtctttctgggttttacatatgtggctccatctgtacttgaaagtgtgaaagaaaagttttcctttgaaccaaaaatccgatcacctcgaagatttattggcagcccacgaacacctgtcagcccagtcaaattttctcctggggatttctggggaagaggtgcttcggccagcacagcaaatcctcagacacctgtggaatacccaatggaaacaagtggcatagagcagatggatgtgacaatgagtggggaagcatcggcaccacttccaatacgacagccgaactctgggccatacaaaaaacaagcttttcccatgatctccaaacggccagagcacctgcgtatgaatctatgacagagcaatgcttttaatgaatttaaggcaaaaaggtggagagggagatgtgtgagcatcctgcaaggtgaaacaagactcaaaatgacagtttcagagagtcaatgtcattacatagaacacttcggacacaggaaaaataaacgtggattttaaaaaatcaatcaatggtgcaaaaaaaaacttaaagcaaaatagtattgctgaactcttaggcacatcaattaattgattcctcgcgacatctttctcaaccttatcaaggattttcatgttgatgactcgaaactgacagtattaagggtaggatgttgctctgaatcactgtgagtctgatgtgtgaagaagggtatcctttcattaggcaagtacaaattgcctataatacttgcaactaaggacaaattagcatgcaagcttggtcaaacttttcccaggcaaaatgggaaggcaaagacaaaagaaacttaccaattgatgttttacgtgcaaacaacctgaatcttttttttatataaatatatatttttcaaatagatttttgattcagctcattatgaaaaacatcccaaactttaaaatgcgaaattattggttggtgtgaagaaagccagacaacttctgtttcttctcttggtgaaataataaaatgcaaatgaatcattgttaacacagctgtggctcgtttgagggattggggtggacctggggtttattttcagtaacccagctgcggagcct

SEQ ID NO: 2 - protein, 525 aa, Homo sapiens
MRRRRRRDGFYPAPDFRDREAEDMAGVFDIDLDQPEDAGSEDELEEGGQLNESMDHGGVGPYELGMEHCEKFEISETSVNRGPEKIRPECFELLRVLGKGGYGKVFQVRKVTGANTGKIFAMKVLKKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDSTLSESANQVFLGFTYVAPSVLESVKEKFSFEPKIRSPRRFIGSPRTPVSPVKFSPGDFWGRGASASTANPQTPVEYPMETSGIEQMDVTMSGEASAPLPIRQPNSGPYKKQAFPMISKRPEHLRMNL

SEQ ID NO: 3 - DNA, 8765 bp, Homo sapiens
  CDS (403)..(7647): p300 acetyltransferase
ggacagcgccgaggaggaagaggttgatggcggcggcggagctccgagagacctcggctgggcaggggccggccgtggcgggccggggactgcgcctctagagccgcgagttctcgggaattcgccgcagcggacgcgctcggcgaatttgtgctcttgtgccctcctccgggcttgggcccaggcccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggatgaatgcgggcatgaatcctggaatgttggctgcaggcaatggacaagggataatgcctaatcaagtcatgaacggttcaattggagcaggccgagggcgacagaatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggcctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctaatggccctctacctgacccaagtatgatccgtggcagtgtgccaaaccagatgatgcctcgaataactccacaatctggtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacacctcctgccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaacagcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatactcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctgcagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagcagcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtagtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatagttattatttactggtgcagatggttgacatttttccctattttcctcactttatggaagagttaaaacatttctaaaccagaggacaaaaggggttaatgttactttaaaattacattctatatatatataaatatatataaatatatattaaaataccagttttttttctctgggtgcaaagatgttcattcttttaaaaaatgtttaaaaaaaaaaaaaaactgcctttcttcccctcaagtcaacttttgtgctccagaaaattttctattctgtaagtctgagcgtaaaacttcaagtattaaaataatttgtacatgtagagagaaaaatgactttttcaaaaatatacaggggcagctgccaaattgatgtattatatattgtggtttctgtttcttgaaagaatttttttcgttatttttacatctaacaaagtaaaaaaattaaaaagagggtaagaaacgattccggtgggatgattttaacatgcaaaatgtccctgggggtttcttctttgcttgctttcttcctccttaccctaccccccactcacacacacacacacacacacacacacacacacacacacacactttctataaaacttgaaaatagcaaaaaccctcaactgttgtaaatcatgcaattaaagttgattacttataaatatgaactttggatcactgtatagactgttaaatttgatttcttattacctattgttaaataaactgtgtgagacag

SEQ ID NO: 4 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 5 - DNA, 1302 bp, Homo sapiens
  CDS (1)..(1302): Active domain of p300 acetyltransferase (p300-4)
aagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagct

SEQ ID NO: 6 - protein, 434 aa, Homo sapiens
KPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQA

SEQ ID NO: 7 - DNA, 1816 bp, Homo sapiens
  CDS (77)..(1561): p70(beta) S6 Kinase gene
gagagagagagagagagagagagagagagagagagagagagagagagagagagagagagagagagactcgtgccgaatggcacgaggccgacgggcccgcggggccggcgccgccatggcggccgtgtttgatttggatttggagacggaggaaggcagcgagggcgagggcgagccagagctcagccccgcggacgcatgtccccttgccgagttgagggcagctggcctagagcctgtgggacactatgaagaggtggagctgactgagaccagcgtgaacgttggcccagagcgcatcgggccccactgctttgagctgctgcgtgtgctgggcaaggggggctatggcaaggtgttccaggtgcgaaaggtgcaaggcaccaacttgggcaaaatatatgccatgaaagtcctaaggaaggccaaaattgtgcgcaatgccaaggacacagcacacacacgggctgagcggaacattctagagtcagtgaagcacccctttattgtggaactggcctatgccttccagactggtggcaaactctacctcatccttgagtgcctcagtggtggcgagctcttcacgcatctggagcgagagggcatcttcctggaagatacggcctgcttctacctggctgagatcacgctggccctgggccatctccactcccagggcatcatctaccgggacctcaagcccgagaacatcatgctcagcagccagggccacatcaaactgaccgactttggactctgcaaggagtctatccatgagggcgccgtcactcacaccttctgcggcaccattgagtacatggcccctgagattctggtgcgcagtggccacaaccgggctgtggactggtggagcctgggggccctgatgtacgacatgctcactggatcgccgccctttaccgcagagaaccggaagaaaaccatggataagatcatcaggggcaagctggcactgcccccctacctcaccccagatgcccgggaccttgtcaaaaagtttctgaaacggaatcccagccagcggattgggggtggcccaggggatgctgctgatgtgcagagacatccctttttccggcacatgaattgggacgaccttctggcctggcgtgtggacccccctttcaggccctgtctgcagtcagaggaggacgtgagccagtttgatacccgcttcacacggcagacgccggtggacagtcctgatgacacagccctcagcgagagtgccaaccaggccttcctgggcttcacatacgtggcgccgtctgtcctggacagcatcaaggagggcttctccttccagcccaagctgcgctcacccaggcgcctcaacagtagcccccgggtccccgtcagccccctcaagttctccccttttgaggggtttcggcccagccccagcctgccggagcccacggagctacctctacctccactcctgccaccgccgccgccctcgaccaccgcccctctccccatccgtcccccctcagggaccaagaagtccaagaggggccgtgggcgtccagggcgctaggaagccgggtgggggtgagggtagcccttgagccctgtccctgcggctgtgagagcagcaggaccctgggccagttccagagacctgggggtgtgtctgggggtggggtgtgagtgcgtatgaaagtgtgtgtctgctggggcagctgtgcccctgaatcatgggcacggagggccgcccgccacaccccgcgctcaactgctcccgtggaagattaaagggctgaatcatgaaaaaaaaaaaaaaaaaaaa

SEQ ID NO: 8 - protein, 495 aa, Homo sapiens
MARGRRARGAGAAMAAVFDLDLETEEGSEGEGEPELSPADACPLAELRAAGLEPVGHYEEVELTETSVNVGPERIGPHCFELLRVLGKGGYGKVFQVRKVQGTNLGKIYAMKVLRKAKIVRNAKDTAHTRAERNILESVKHPFIVELAYAFQTGGKLYLILECLSGGELFTHLEREGIFLEDTACFYLAEITLALGHLHSQGIIYRDLKPENIMLSSQGHIKLTDFGLCKESIHEGAVTHTFCGTIEYMAPEILVRSGHNRAVDWWSLGALMYDMLTGSPPFTAENRKKTMDKIIRGKLALPPYLTPDARDLVKKFLKRNPSQRIGGGPGDAADVQRHPFFRHMNWDDLLAWRVDPPFRPCLQSEEDVSQFDTRFTRQTPVDSPDDTALSESANQAFLGFTYVAPSVLDSIKEGFSFQPKLRSPRRLNSSPRVPVSPLKFSPFEGFRPSPSLPEPTELPLPPLLPPPPPSTTAPLPIRPPSGTKKSKRGRGRPGR

SEQ ID NO: 9 - protein, 14 aa, Artificial
  Synthetic peptide
QAFPMISKRPEHLR

SEQ ID NO: 10 - protein, 14 aa, Artificial
  Synthetic peptide
GKLALPPYLTPDAR

SEQ ID NO: 11 - protein, 14 aa, Artificial
  Synthetic peptide
VLGKGGYGKVFQVR

SEQ ID NO: 12 - protein, 9 aa, Artificial
  Synthetic peptide
KRKRGRPRK

SEQ ID NO: 13 - protein, 9 aa, Artificial
  Synthetic peptide
KRGRGRPGR

SEQ ID NO: 14 - protein, 9 aa, Artificial
  Synthetic peptide
KRGRGRPRG

SEQ ID NO: 15 - protein, 8 aa, Artificial
  Synthetic peptide
KGRGRPKK

SEQ ID NO: 16 - protein, 8 aa, Artificial
  Synthetic peptide
RTRGRPAD

SEQ ID NO: 17 - protein, 9 aa, Artificial
  Synthetic peptide
KRKRGRPRK

SEQ ID NO: 18 - protein, 9 aa, Artificial
  Synthetic peptide
KRGRGRPRL

SEQ ID NO: 19 - protein, 9 aa, Artificial
  Synthetic peptide
KRVRGRPCL

SEQ ID NO: 20 - protein, 9 aa, Artificial
  Synthetic peptide
KKSRGRPKK

SEQ ID NO: 21 - protein, 9 aa, Artificial
  Synthetic peptide
KRRRGRPPK

SEQ ID NO: 22 - protein, 9 aa, Artificial
  Synthetic peptide
KRGRGRPKG

SEQ ID NO: 23 - protein, 9 aa, Artificial
  Synthetic peptide
GRGRGRPKG

SEQ ID NO: 24 - protein, 9 aa, Artificial
  Synthetic peptide
KRGRGRPRK

SEQ ID NO: 25 - protein, 9 aa, Artificial
  Synthetic peptide
KRGRGRPGR

SEQ ID NO: 26 - protein, 9 aa, Artificial
  Synthetic peptide
KKGRGRPGR

SEQ ID NO: 27 - protein, 9 aa, Artificial
  Synthetic peptide
KRGRGRPGR

SEQ ID NO: 28 - protein, 9 aa, Artificial
  Synthetic peptide
KRGRGRPGR

SEQ ID NO: 29 - protein, 9 aa, Artificial
  Synthetic peptide
KRGRGRPGR